SEQUENCE LISTING (3 sequences)

SEQ ID NO: 1 - DNA, 11 bp, synthetic construct
  source 1..11: mol_type = other DNA
agtaccgtacc

SEQ ID NO: 2 - DNA, 11 bp, synthetic construct
  source 1..11: mol_type = other DNA
tagcgaagcga

SEQ ID NO: 3 - DNA, 11 bp, synthetic construct
  source 1..11: mol_type = other DNA
agtagaagcga

(19) United States
(12) Patent Application Publication
(10) Pub. No.: US 2024/0185953 A1
(43) Pub. Date: Jun. 6, 2024

(54) SYSTEMS AND METHODS FOR HIGH-THROUGHPUT PREDICTIONS

(71) Applicant: Regeneron Pharmaceuticals, Inc., Tarrytown, NY (US)

(72) Inventors: Wen Fury, Tarrytown, NY (US); Akshaya Raghavan, Tarrytown, NY (US)

(21) Appl. No.: 18/526,599

(22) Filed: Dec. 1, 2023

Related U.S. Application Data
(60) Provisional application No. 63/429,949, filed on Dec. 2, 2022.

(51) Int. Cl. G16B 20/40

(52) U.S. Cl. CPC G16B 20/40

(57) ABSTRACT

Systems and methods for predicting a prevalence of loss of heterozygosity (LOH) in a target population of cells are disclosed, wherein the system comprises a memory and a processor configured to receive genetic data for a first reference population of cells. The processor is configured to sequence the genetic data for the first reference population of cells to obtain first reference data; identify and remove heterozygous variant positions having imbalanced allelic expression in the first reference data to generate second reference data; map identifiers for each cell of the target population of cells to the second reference data; and apply the mapped identifiers for each cell of the target population of cells to a supervised machine learning model. The processor is further configured to receive one or more outputs from the model, at least one of the one or more outputs including an LOH for the target population of cells.

### CROSS REFERENCE TO RELATED APPLICATIONS

This application claims priority to U.S. Provisional Patent Application No. 63/429,949, filed on Dec. 2, 2022, which is incorporated by reference herein in its entirety.

### REFERENCE TO SEQUENCE LISTING

This application includes a Sequence Listing filed electronically as an XML file named 381204005SEQ, created on Dec. 1, 2023, with a size of 3,506 bytes. The Sequence Listing is incorporated herein by reference.

### FIELD

This application relates generally to predictive modeling and, more particularly, to systems and methods for prediction of loss of heterozygosity (LOH).

### BACKGROUND

Existing systems and methods for prediction of loss of heterozygosity (LOH) are labor intensive, time consuming and may not be applicable to all cells. The overall loss of heterozygosity rate is low, in the reported range of about 5-6%. There is currently no high throughput method available to accurately assess the loss of heterozygosity rate. Therefore, there is a need for a high throughput method to accurately assess the loss of heterozygosity rate.

### SUMMARY

Existing systems and methods for zygosity assessment include single nucleotide polymorphism (SNP) array combined with array comparative genomic hybridization (aCGH), SNP genotyping combined with fluorescence in situ hybridization (FISH), single-cell DNA sequencing (scDNAseq), bulk DNA sequencing (DNAseq) and bulk RNA sequencing (RNAseq)-based assessments (Boutin, et al., Nature Communications, 2021; Alanis-Lobato, et al., PNAS, 2021; Groff, et al., Genome Research, 2019; Groff, et al., Genome Research, 2019). However, limitations associated with the known methods for zygosity assessment include a cell cloning requirement, low throughput, cost and low resolution. Challenges associated with single-cell RNA sequencing include determining the minimum unique molecular identifier (UMI) coverage requirement to be included in zygosity assessment for a single variant position, how to perform zygosity measurement for a DNA segment of a cell with multiple variant positions, the threshold for whether zygosity is assessable for a cell due to overall unique molecular identifier coverage, and frequency of sequencing errors.

In one aspect, the present disclosure relates to high throughput methods for inferring loss of heterozygosity in individual cells using single-cell RNA sequencing (inferLOH). In another embodiment, the present disclosure also relates to high throughput methods for detecting the prevalence of loss of heterozygosity in a cell population by inferring the zygosity of the chromosome region in which there is potentially a loss of heterozygosity (LOHR) at the single-cell level. A benefit afforded by the present disclosure is a system and method for assessing loss of heterozygosity (LOH) using single cell RNA sequencing that overcomes the limitations commonly associated with single cell RNA sequencing, which include, for example, high dropout rate, low coverage, and sequencing errors. The system and method also overcomes the potential shortcomings of using observed RNA sequences to infer DNA zygosity, for instance, allelic expression imbalance or RNA editing.

For example, in one aspect, the systems and methods of the present disclosure can predict loss of heterozygosity cells with over 99% sensitivity and 99% specificity. Another benefit afforded by the present disclosure is that a population of pure loss of heterozygosity cells, which may not be available, are not needed as part of the training data to build the model for determining loss of heterozygosity. Consequently, the present disclosure provides methods that fulfill the long-felt need for an accurate, high-throughput method of examining loss of heterozygosity at the single-cell level, as well as within a population of cells, while also circumventing the necessity for loss of heterozygosity cells that may not be available and are difficult to produce.

In various aspects, systems for predicting a prevalence of loss of heterozygosity (LOH) in a target population of cells is provided.

In some aspects, the system may include at least one memory storing computer-executable instructions and at least one processor in communication with the at least one memory. In some aspects, the at least one processor may be configured to execute the computer-executable instructions to: receive genetic data for a first reference population of cells; sequence the genetic data for the first reference population of cells to obtain first reference data, the first reference data comprising chromosome identification, nucleotide coordinates, nucleotide composition, or combinations thereof; identify and remove heterozygous variant positions having imbalanced allelic expression in the first reference data to generate second reference data; map identifiers for each cell of the target population of cells to the second reference data; apply one or more inputs to a supervised machine learning model, the one or more inputs comprising the mapped identifiers for each cell of the target population of cells, the model being previously trained using historical data, the historical data comprising mapped identifiers for each cell of the target population of cells and their corresponding LOH; and receive one or more outputs from the model, at least one of the one or more outputs including an LOH for the target population of cells, thereby predicting the prevalence of LOH in the target population of cells; update the historical data to include the genetic data for the target population of cells and the corresponding one or more outputs; and re-train the model using the updated historical data.

In some aspects, the at least one processor may be configured to execute the computer-executable instructions to: receive genetic data for a first reference population of cells; sequence the genetic data for the first reference population of cells to obtain first reference data, the first reference data comprising chromosome identification, nucleotide coordinates, nucleotide composition, or combinations thereof; identify and remove heterozygous variant positions having imbalanced allelic expression in the first reference data based on single cell RNA sequencing data generated from a second reference population of cells to generate second reference data; establish a set of homozygous positions that are a certain number of nucleotide distances away from each variant position of the second reference data to generate third reference data; map identifiers for each cell of the target population of cells to the second reference data; map identifiers for each cell of the second reference population of cells to the second reference data and/or the third reference data; apply one or more inputs to a supervised machine learning model, the one or more inputs comprising the mapped identifiers for each cell of the target population of cells and the mapped identifiers for each cell of the second reference population of cells, the model being previously trained using historical data, the historical data comprising mapped identifiers for each cell of the target population of cells and their corresponding LOH; and receive one or more outputs from the model, at least one of the one or more outputs including an LOH for the target population of cells, thereby predicting the prevalence of LOH in the target population of cells; update the historical data to include the genetic data for the target population of cells and the corresponding one or more outputs; and re-train the model using the updated historical data.

The systems may include additional, less, or alternate functionality, including that discussed elsewhere herein.

In various aspects, computer-implemented methods for predicting a prevalence of loss of LOH in a target population of cells is provided. The methods may be implemented using a system including a computing device including a processor communicatively coupled to a memory device. Additionally, or alternatively, the computer-implemented methods may be implemented via one or more local or remote processors, servers, transceivers, memory units, mobile devices, wearables, smart watches, smart contact lenses, smart glasses, augmented reality glasses, virtual reality headsets, mixed or extended reality glasses or headsets, voice or chat bots, ChatGPT bots, and/or other electronic or electrical components, which may be in wired or wireless communication with one another.

In some aspects, the method may comprise receiving genetic data for a first reference population of cells; sequencing the genetic data to obtain first reference data, the first reference data comprising chromosome identification, nucleotide coordinates, nucleotide composition, or combinations thereof; identifying and removing heterozygous variant positions having imbalanced allelic expression in the first reference data to generate second reference data; mapping identifiers for each cell of the target population of cells to the second reference data; applying one or more inputs to a supervised machine learning model, the one or more inputs comprising the mapped identifiers for each cell of the target population of cells, the model being previously trained using historical data, the historical data comprising mapped identifiers for each cell of the target population of cells and their corresponding LOH; and receiving one or more outputs from the model, at least one of the one or more outputs including an LOH for the target population of cells, thereby predicting the prevalence of LOH in the target population of cells; updating the historical data to include the genetic data for the target population of cells and the corresponding one or more outputs; and re-training the model using the updated historical data.

In some aspects, the method may comprise receiving genetic data for a first reference population of cells; sequencing the genetic data for the first reference population of cells to obtain first reference data, the first reference data comprising chromosome identification, nucleotide coordinates, nucleotide composition, or combinations thereof; identifying and removing heterozygous variant positions having imbalanced allelic expression in the first reference data based on single cell RNA sequencing data generated from a second reference population of cells to generate second reference data; establishing a set of homozygous positions that are a certain number of nucleotide distances away from each variant position of the second reference data to generate third reference data; mapping identifiers for each cell of the target population of cells to the second reference data; mapping identifiers for each cell of the second reference population of cells to the second reference data and/or the third reference data; applying one or more inputs to a supervised machine learning model, the one or more inputs comprising the mapped identifiers for each cell of the target population of cells and the mapped identifiers for each cell of the second reference population of cells, the model being previously trained using historical data, the historical data comprising mapped identifiers for each cell of the target population of cells and their corresponding LOH; and receiving one or more outputs from the model, at least one of the one or more outputs including an LOH for the target population of cells, thereby predicting the prevalence of LOH in the target population of cells; updating the historical data to include the genetic data for the target population of cells and the corresponding one or more outputs; and re-training the model using the updated historical data.

The methods may include additional, less, or alternate functionality, including those discussed elsewhere herein.

In various aspects, at least one non-transitory computer-readable storage media having computer-executable instructions embodied thereon is provided. In some aspects, the computer-executable instructions, when executed by at least one processor, may cause the at least one processor to: receive genetic data for a first reference population of cells; sequence the genetic data for the first reference population of cells to obtain first reference data, the first reference data comprising chromosome identification, nucleotide coordinates, nucleotide composition, or combinations thereof; identify and remove heterozygous variant positions having imbalanced allelic expression in the first reference data to generate second reference data; map identifiers for each cell of the target population of cells to the second reference data; apply one or more inputs to a supervised machine learning model, the one or more inputs comprising the mapped identifiers for each cell of the target population of cells, the model being previously trained using historical data, the historical data comprising mapped identifiers for each cell of the target population of cells and their corresponding LOH; and receive one or more outputs from the model, at least one of the one or more outputs including an LOH for the target population of cells, thereby predicting the prevalence of LOH in the target population of cells; update the historical data to include the genetic data for the target population of cells and the corresponding one or more outputs; and re-train the model using the updated historical data.

In some aspects, the computer-executable instructions, when executed by at least one processor, may cause the at least one processor to: receive genetic data for a first reference population of cells; sequence the genetic data for the first reference population of cells to obtain first reference data, the first reference data comprising chromosome identification, nucleotide coordinates, nucleotide composition, or combinations thereof; identify and remove heterozygous variant positions having imbalanced allelic expression in the first reference data to generate second reference data; establish a set of homozygous positions that are a certain number of nucleotide distances away from each variant position of the second reference data to generate third reference data; map identifiers for each cell of the target population of cells to the second reference data; map identifiers for each cell of the second reference population of cells to the second reference data and/or the third reference data; apply one or more inputs to a supervised machine learning model, the one or more inputs comprising the mapped identifiers for each cell of the target population of cells and the mapped identifiers for each cell of the second reference population of cells, the model being previously trained using historical data, the historical data comprising mapped identifiers for each cell of the target population of cells and their corresponding LOH; and receive one or more outputs from the model, at least one of the one or more outputs including an LOH for the target population of cells, thereby predicting the prevalence of LOH in the target population of cells; update the historical data to include the genetic data for the target population of cells and the corresponding one or more outputs; and re-train the model using the updated historical data.

The storage medium may include additional, less, or alternate functionality, including that discussed elsewhere herein.

In some aspects, the systems and methods of the disclosure allow for predicting a prevalence of loss of heterozygosity (LOH) in a target population of cells wherein no cell cloning is required.

In some aspect, the systems and methods provide a unique molecular identifier instead of conventional read count-based zygosity calling.

In some aspects, the systems and methods provide for machine learning-based loss of heterozygosity prediction. In some aspects, the systems and methods do not require “purified” loss of heterozygosity cells to train the machine learning based model. Instead, mock loss of heterozygosity (mLOH) cell data can be derived from wild-type (WT) cells.

In some aspects, the systems and methods provide a prediction model of LOH for individual cells while also minimizing sampling error.

In some aspects, the systems and methods are cell type, CRISPR editing site, and 10x genomics single-cell RNA sequencing platform-independent.

Challenges in developing a model for detecting loss of heterozygosity include: if a model is developed based on current data (cell type, CRISPR editing site, and 10x Genomics), it may not be applicable for other CRISPR editing sites, different cell types, or single-cell RNA sequencing data generated form a different technology; pure loss of heterozygosity cells may not be available to train the prediction model; and there may be gene allelic expression imbalance.

In some aspects, the present disclosure also provides systems and methods for predicting a prevalence of loss of heterozygosity (LOH) in a population of cells edited by CRISPR comprising: bulk DNA sequencing a first population of cells having the same DNA genotype as the WT cells to obtain a heterozygous reference (HetRef) comprising chromosome identification, nucleotide coordinates, nucleotide composition, or combinations thereof; identifying heterozygous variant positions having imbalanced allelic expression in the HetRef based on single cell RNA sequencing data generated from WT cells and removing heterozygous variant positions having imbalanced allelic expression in the HetRef to generate a

HetRef2; mapping UMI from each cell in the testing sample to a reference genome and to generate a UMI coverage correlated to the HetRef2 coordinates, and calculating a zygosity score based on the UMI coverage related to the two nucleotides registered in each HetRef2 position; generating a range of the UMI coverage threshold; and predicting the prevalence of LOH in a population of cells edited by CRISPR based on a percentage of cells predicted to be LOH using a model, the UMI coverage threshold, or combinations thereof.

In some aspects, the present disclosure provides systems and methods wherein a variant position is included in HetRef if it is covered by at least about 20 DNA sequencing reads.

In some aspects, the present disclosure provides systems and methods wherein heterozygous variant positions having imbalanced allelic expression are defined if 80% or more of UMIs mapped to a HetRef position are covered by the same nucleotide.

In some aspects, the present disclosure provides systems and methods wherein the scRNAseq data are excluded for the calculation of a zygosity score if they do not meet a minimum threshold for number of UMIs detected at a HetRef position.

In some aspects, the present disclosure provides systems and methods wherein a position is considered heterozygous if each one of the two alleles in bulk DNA sequencing is represented by between about 20% and about 80% of reads in a DNA sequencing dataset.

In one aspect of the present disclosure, bulk DNA sequencing refers to random sequencing of multiple cells in a mixture of pooled cells. In one aspect of the present disclosure, bulk RNA sequencing refers to random sequencing of pooled cells.

In some aspects, the present disclosure provides systems and methods wherein the HetRef2 is a subset of the HetRef.

In some aspects, the present disclosure provides systems and methods wherein the HetRef2 removes any heterozygous variant positions having imbalanced allelic expression from the HetRef.

In some aspects, the present disclosure provides systems and methods wherein the minimum threshold is at least 4 UMI.

In some aspects, the present disclosure provides systems and methods wherein the systems and methods are used to determine LOH in cancer cells.

In some aspects, the present disclosure further provides systems and methods for predicting the prevalence of loss of heterozygosity (LOH) in a population of cells edited by CRISPR comprising: DNA sequencing bulk DNA from a first population of cells having heterozygosity in a DNA segment of interest to obtain a first heterozygous variant reference (HetRef) comprising chromosome coordinates and nucleotide composition; single cell RNA sequencing cells from a second population of cells to identify and remove allelic expression imbalanced HetRef in the first heterozygous variant reference, to generate a second heterozygous variant reference (HetRef2); generating a homozygous variant reference (HomRef) by establishing a set of homozygous positions that are a certain number of nucleotide distances from each variant position in HetRef2; using unique molecular identifiers (UMI) generated from single-cell RNA sequencing cells from the second population of wild-type (WT) cells mapped to HetRef2 or HomRef coordinates to train a model as WT cells or mock LOH (mLOH) cells for detecting LOH; performing a coverage and zygosity assessment wherein coverage is equal to a sum of all UMIs covering the HetRef2 or HomRef coordinates and the zygosity score is equal to a sum of UMI of the less frequently covered allele of each position across all coordinates registered in HetRef or HomRef divided by the respective coverage; single cell RNA sequencing cells from a population of cells of known genotype (WT or LOH) to validate the model for detecting LOH; and predicting the prevalence of LOH in a cell population of interest based on the model for detecting LOH.

In some aspects, the present disclosure provides systems and methods wherein a variant position is only included in HetRef if it is covered by at least about 20 DNA sequencing reads. In some aspects, the present disclosure provides systems and methods wherein heterozygous variant positions having imbalanced allelic expression are defined if 80% or more of UMIs mapped to a HetRef position are covered by the same nucleotide.

In some aspects, the present disclosure provides systems and methods wherein the scRNAseq data are excluded for the calculation of a zygosity score if they do not meet a minimum threshold for number of UMIs detected for a HetRef position.

In some aspects, the present disclosure provides systems and methods wherein a position is considered heterozygous if each one of the two alleles in bulk DNA sequencing is represented by between about 20% and about 80% of reads in a DNA sequencing dataset.

In some aspects, the present disclosure provides systems and methods wherein the HetRef2 is a subset of the HetRef.

In some aspects, the present disclosure provides systems and methods wherein the HetRef2 removes any allelic expression imbalanced variant positions from the HetRef.

In some aspects, the present disclosure provides systems and methods wherein the minimum threshold is at least 4 UMI.

In some aspects, the present disclosure provides systems and methods wherein the method is used to determine the LOH in cancer cells.

In yet another aspect, the present disclosure provides systems and methods for predicting the prevalence of loss of heterozygosity (LOH) in a population of cells edited by CRISPR comprising: bulk DNA sequencing a first population of cells having heterozygosity in a DNA segment of interest to obtain a heterozygous reference (HetRef) comprising chromosome identification, nucleotide coordinates, and nucleotide composition; single cell RNA sequencing a second population of cells untreated with CRISPR (WT) to obtain a dataset comprising a plurality of unique molecular identifiers (UMI), and the sequence of each UMI and its map to a chromosomal location; single cell RNA sequencing a third population of cells (testing sample) comprising CRISPR-treated cells that may include cells that have lost heterozygosity induced by the CRISPR procedure, the sequence of each UMI and its map to a chromosomal location from the testing sample; identifying heterozygous variant positions having imbalanced allelic expression in the HetRef based on single cell RNA sequencing data generated from the second population of WT sample cells and removing allelic expression imbalanced positions in the HetRef to generate a HetRef2; generating homozygous variant reference (HomRef) based on HetRef2; mapping UMI from each cell in the WT sample to the HetRef2 to generate a UMI coverage, and calculating a WT zygosity score based on the UMI coverage; mapping UMI from each cell in the WT sample to the HomRef to generate a UMI coverage, and calculating a mock loss of heterozygosity (mLOH) zygosity score based on the UMI coverage; mapping UMI from each cell in the testing sample to the HetRef2 to generate a UMI coverage, and calculating a zygosity score based on the UMI coverage; generating a range of the UMI coverage threshold; generating a zygosity prediction model instance using WT and mLOH zygosity score generated from WT or mLOH cells that meet each UMI coverage threshold in the threshold range; calculating the number of cells in the testing sample that meet each UMI coverage threshold; selecting the best model based on the model performance as well as the number of cells in the testing sample meet the corresponding UMI coverage threshold to ensure model accuracy and minimizing sampling error in the testing sample; and predicting the prevalence of LOH in the cells of the testing sample based on a percentage of cells predicted to be LOH using the best model and the UMI coverage threshold. In some aspects, the present disclosure provides systems and methods wherein a variant position is included in HetRef if it is covered by at least about 20 DNA sequencing reads.

In some aspects, the present disclosure provides systems and methods wherein heterozygous variant positions having imbalanced allelic expression are defined if 80% or more of UMIs mapped to a HetRef position are covered by the same nucleotide.

In some aspects, the present disclosure provides systems and methods wherein the scRNAseq data are excluded for the calculation of a zygosity score if they do not meet a minimum threshold for number of UMIs detected for a HetRef position.

In some aspects, the present disclosure provides systems and methods wherein a position is considered heterozygous if each one of the two alleles in bulk DNA sequencing is represented by between about 20% and about 80% of reads in a DNA sequencing dataset.

In some aspects, the present disclosure provides systems and methods wherein the HetRef2 is a subset of the HetRef.

In some aspects, the present disclosure provides systems and methods wherein the HetRef2 removes any allelic expression imbalanced variant positions from the HetRef.

In some aspects, the present disclosure provides systems and methods wherein the minimum threshold is at least 4 UMI.

In some aspects, the present disclosure provides systems and methods wherein the systems and methods are used to determine the LOH in cancer cells.

In various aspects, the present disclosure provides systems and methods for predicting the prevalence of loss of heterozygosity (LOH) in a population of cells comprising: DNA sequencing bulk DNA from a first population of cells having heterozygosity in a DNA segment of interest to obtain a first heterozygous variant reference (HetRef) comprising chromosome coordinates and nucleotide composition; single-cell RNA sequencing cells from the second population of cells to identify and remove allelic expression imbalanced HetRef in the first heterozygous variant reference, to generate a second heterozygous variant reference (HetRef2); single cell RNA sequencing cells from a second population of cells with loss of heterozygosity to generate a mock loss of heterozygosity (mLOH) homozygous variant reference (HomRef) for training a model for detecting loss of heterozygosity; single cell RNA sequencing wild-type cells and mLOH cells and performing a coverage and zygosity assessment wherein coverage is equal to a sum of all UMIs covering HetRef2 or HomRef coordinates and zygosity score is equal to a sum of UMI of the less frequently covered allele of each position across all coordinates registered in HetRef or HomRef divided by the respective coverage; single cell RNA sequencing cells from a population of cells of known genotype (WT or LOH) to validate the model for detecting LOH; and predicting the prevalence of LOH in a cell population of interest based on the model for detecting LOH.

In some aspects, the present disclosure provides systems and methods wherein a variant position is included in HetRef if it is covered by at least about 20 DNA sequencing reads.

In some aspects, the present disclosure provides systems and methods wherein heterozygous variant positions having imbalanced allelic expression are defined if 80% or more of UMIs mapped to a HetRef position are covered by the same nucleotide.

In some aspects, the present disclosure provides systems and methods wherein the scRNAseq data are excluded for the calculation of a zygosity score if they do not meet a minimum threshold for number of UMIs detected for a HetRef position.

In some aspects, the present disclosure provides systems and methods wherein a position is considered heterozygous if each one of the two alleles in bulk DNA sequencing is represented by between about 20% and about 80% of reads in a DNA sequencing dataset.

In some aspects, the present disclosure provides systems and methods wherein the HetRef2 is a subset of the HetRef.

In some aspects, the present disclosure provides systems and methods wherein the HetRef2 removes any allelic expression imbalanced variant positions from the HetRef.

In some aspects, the present disclosure provides systems and methods wherein the minimum threshold is at least 4 UMI.

In some aspects, the present disclosure provides systems and methods wherein the method is used to determine the LOH in cancer cells.

In various aspects, the present disclosure provides systems and methods for predicting the prevalence of loss of heterozygosity (LOH) in a population of cells comprising: single-cell RNA sequencing a cell population of interest and generating a coverage assessment and zygosity score wherein the coverage is equal to a sum of all unique molecular identifiers (UMI) and the zygosity score is equal to a sum of UMI of the less frequently covered allele across all registered coordinates divided by the respective coverage; single cell RNA sequencing a mock loss of heterozygosity population (mLOH) of cells; generating a UMI coverage and a range of the UMI coverage threshold; generating a model for predicting the prevalence of LOH in a cell population based on the single cell RNA sequencing of the cell population of interest and a mLOH population of cells; and predicting the prevalence of LOH in a cell population of interest based on the model and the UMI coverage threshold.

In some aspects, the present disclosure provides systems and methods wherein a variant position is included in HetRef if it is covered by at least about 20 DNA sequencing reads.

In some aspects, the present disclosure provides systems and methods wherein heterozygous variant positions having imbalanced allelic expression are defined if 80% or more of UMIs mapped to a HetRef position are covered by the same nucleotide.

In some aspects, the present disclosure provides systems and methods wherein the scRNAseq data are excluded for the calculation of a zygosity score if they do not meet a minimum threshold for number of UMIs detected for a HetRef position.

In some aspects, the present disclosure provides systems and methods wherein a position is considered heterozygous if each one of the two alleles in bulk DNA sequencing is represented by between about 20% and about 80% of reads in a DNA sequencing dataset.

In some aspects, the present disclosure provides systems and methods wherein the HetRef2 is a subset of the HetRef. In some aspects, the present disclosure provides systems and methods wherein the HetRef2 removes any allelic expression imbalanced variant positions from the HetRef.

In some aspects, the present disclosure provides systems and methods wherein the minimum threshold is at least 4 UMI.

In some aspects, the present disclosure provides systems and methods wherein the systems and methods are used to determine the LOH in cancer cells.

In various aspects, the present disclosure provides systems and methods for predicting a prevalence of loss of heterozygosity (LOH) in the genome of a cancer cell comprising: bulk DNA sequencing a first population of cells having heterozygosity in a DNA segment of interest to obtain a heterozygous reference (HetRef) comprising chromosome identification, nucleotide coordinates, nucleotide composition, or combinations thereof; identifying heterozygous variant positions having imbalanced allelic expression in the HetRef based on single cell RNA sequencing data generated from testing sample cells and removing allelic expression imbalanced positions in the HetRef to generate a HetRef2; mapping UMI from each cell in the testing sample to the HetRef2 coordinates to generate a UMI coverage, and calculating a zygosity score based on the UMI coverage; generating a range of the UMI coverage threshold; and predicting the prevalence of LOH in a population of cells based on a percentage of cells predicted to be LOH using a model, the UMI coverage threshold, or combinations thereof.

In some aspects, the present disclosure provides systems and methods wherein a variant position is included in HetRef if it is covered by at least about 20 DNA sequencing reads.

In some aspects, the present disclosure provides systems and methods wherein heterozygous variant positions having imbalanced allelic expression are defined if 80% or more of UMIs mapped to a HetRef position are covered by the same nucleotide.

In some aspects, the present disclosure provides systems and methods wherein the scRNAseq data are excluded for the calculation of a zygosity score if they do not meet a minimum threshold for number of UMIs detected for a HetRef position.

In some aspects, the present disclosure provides systems and methods wherein a position is considered heterozygous if each one of the two alleles in bulk DNA sequencing is represented by between about 20% and about 80% of reads in a DNA sequencing dataset.

In some aspects, the present disclosure provides systems and methods wherein the HetRef2 is a subset of the HetRef. In some aspects, the present disclosure provides systems and methods wherein the HetRef2 removes any allelic expression imbalanced variant positions from the HetRef.

In some aspects, the present disclosure provides systems and methods wherein the minimum threshold is at least 4 UMI.

In some aspects, the present disclosure provides systems and methods wherein the method is used to determine the LOH in cancer cells.

In various aspects, the present disclosure provides systems and methods for predicting a prevalence of loss of heterozygosity (LOH) in the genome of a naturally-occurring cell comprising: bulk DNA sequencing a first population of cells having heterozygosity in a DNA segment of interest to obtain a heterozygous reference (HetRef) comprising chromosome identification, nucleotide coordinates, nucleotide composition, or combinations thereof; identifying heterozygous variant positions having imbalanced allelic expression in the HetRef based on single cell RNA sequencing data generated from testing sample cells and removing allelic expression imbalanced positions in the HetRef to generate a HetRef2; generating a UMI coverage, and calculating a zygosity score based on the UMI coverage; generating a range of the UMI coverage threshold; and predicting the prevalence of LOH in a population of cells based on a percentage of cells predicted to be LOH using a model, the UMI coverage threshold, or combinations thereof.

In some aspects, the present disclosure provides systems and methods wherein a variant position is included in HetRef if it is covered by at least about 20 DNA sequencing reads.

In some aspects, the present disclosure provides systems and methods wherein heterozygous variant positions having imbalanced allelic expression are defined if 80% or more of UMIs mapped to a HetRef position are covered by the same nucleotide.

In some aspects, the present disclosure provides systems and methods wherein the scRNAseq data are excluded for the calculation of a zygosity score if they do not meet a minimum threshold for number of UMIs detected for a HetRef position.

In some aspects, the present disclosure provides systems and methods wherein a position is considered heterozygous if each one of the two alleles in bulk DNA sequencing is represented by between about 20% and about 80% of reads in a DNA sequencing dataset.

In some aspects, the present disclosure provides systems and methods wherein the HetRef2 is a subset of the HetRef.

In some aspects, the present disclosure provides systems and methods wherein the HetRef2 removes any allelic expression imbalanced variant positions from the HetRef.

In some aspects, the present disclosure provides systems and methods wherein the minimum threshold is at least 4 UMI.

In one aspect, the present disclosure provides a method wherein the method is used to determine the LOH in cancer cells.

The technical effect of the systems and methods described herein may be achieved by performing the following steps: receive genetic data for a first reference population of cells; sequence the genetic data for the first reference population of cells to obtain first reference data, the first reference data comprising chromosome identification, nucleotide coordinates, nucleotide composition, or combinations thereof; identify and remove heterozygous variant positions having imbalanced allelic expression in the first reference data to generate second reference data; map identifiers for each cell of the target population of cells to the second reference data; apply one or more inputs to a supervised machine learning model, the one or more inputs comprising the mapped identifiers for each cell of the target population of cells, the model being previously trained using historical data, the historical data comprising mapped identifiers for each cell of the target population of cells and their corresponding LOH; and receive one or more outputs from the model, at least one of the one or more outputs including an LOH for the target population of cells, thereby predicting the prevalence of LOH in the target population of cells; update the historical data to include the genetic data for the target population of cells and the corresponding one or more outputs; and re-train the model using the updated historical data

At least one of the technical problems addressed by the systems and methods disclosed herein may include: (i) limitations associated with the known methods for zygosity assessment, which include a cell cloning requirement, low throughput, cost and low resolution; (ii) challenges associated with single-cell RNA sequencing, including determining the minimum unique molecular identifier (UMI) coverage requirement to be included in zygosity assessment for a single variant position, how to perform zygosity measurement for a DNA segment of a cell with multiple variant positions, the threshold for whether zygosity is assessable for a cell due to overall unique molecular identifier coverage, and frequency of sequencing errors.

The resulting technical effects may include: (i) overcoming the limitations commonly associated with single cell RNA sequencing, which include, for example, high dropout rate, low coverage, and sequencing errors; (ii) overcoming the potential shortcomings of using observed RNA sequences to infer DNA zygosity, for instance, allelic expression imbalance or RNA editing; (iii) ability to predict loss of heterozygosity cells with over 99% sensitivity and 99% specificity; (iv) ability to predict loss of heterozygosity without the need for a population of pure loss of heterozygosity cells; (v) fulfilling the long-felt need for an accurate, high-throughput method of examining loss of heterozygosity at the single-cell level, as well as within a population of cells, while also circumventing the necessity for loss of heterozygosity cells that may not be available and are difficult to produce.

These, and other aspects of the present disclosure, will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the disclosure.

### BRIEF DESCRIPTION OF THE DRAWINGS

FIG. 1 illustrates a block diagram of an example computer system in accordance with an embodiment of the present disclosure.

FIG. 2 illustrates a block diagram of a loss of heterozygosity (LOH) prediction computing device that may be used with the example computer system illustrated in FIG. 1.

FIG. 3 illustrates a block diagram of an artificial intelligence (AI)/deep learning (DL) module that may be used with the LOH prediction computing device illustrated in FIG. 2.

FIG. 4 illustrates an example of CRISPR editing-induced LOH, according to an example embodiment. Only heterozygous coordinates based on WT cells are illustrated.

FIG. 5 illustrates an example of how most unique molecular identifiers (UMIs) that are derived from single-cell RNA sequencing and map to a chromosome region in which there is potentially a loss of heterozygosity (LOHR) do not cover heterozygous variant positions registered in HetRef (or HetRef2), and that the coverage of heterozygous variant positions by UMIs that derive from single-cell RNA sequencing is generally low, according to an example embodiment.

FIG. 6 shows the distribution of the heterozygous variant positions on chromosome 16 of wild-type (top panel) or LOH (bottom panel) cells that were identified using single-cell RNA sequencing and the first heterozygous variant reference as a reference, according to an example embodiment.

FIG. 7 shows the histogram of total number of UMIs covering the heterozygous variant positions of HetRef2 in the LOHR of each wild-type cell (top panel), the histogram of total number of heterozygous variant positions of HetRef2 covered by UMIs in the LOHR of each wild-type cell (middle panel) and coverage of heterozygous variant positions of HetRef2 by UMIs in the LOHR of all wild-type cells, according to an example embodiment.

FIG. 8 illustrates how zygosity information from all eligible heterozygous variant positions of HetRef2 in the LOHR were compiled to obtain the overall zygosity score and UMI coverage for each cell, according to an example embodiment.

FIG. 9 shows the sum of unique molecular identifier coverage by at least 4 UMIs per heterozygous variant position of HetRef2 in the LOHR in chromosome 16 of wild-type cells (top panel) and the number of heterozygous variant positions of HetRef2 covered by at least 4 UMIs in the LOHR in chromosome 16 of wild-type cells, according to an example embodiment.

FIG. 10 illustrates how the homozygous reference (HomRef) including the homozygous positions within ±100 chromosome coordinates of each heterozygous variant position in HetRef2 of wild-type cells was generated, and illustrates how mock LOH (mLOH) cells can be generated from WT cells by focusing on HomRef coordinates, according to an example embodiment.

FIG. 11 illustrates how the impurity information from all eligible heterozygous variant positions of HetRef2 in the LOHR of wild-type cells, LOH cells or mLOH cells were compiled to form coverage and zygosity scores for each cell, according to an example embodiment.

FIG. 12 shows a histogram of the sum of the UMIs covering the HetRef2 coordinates in the LOHR on chromosome 16 of wild-type cells with at least 4 UMIs for each variant position, according to an example embodiment.

FIG. 13 illustrates that coverage requirement for the minimum number of UMIs can affect model accuracy and sampling error in the opposite ways, according to an example embodiment.

FIG. 14 illustrates how dynamically determining the coverage requirement for determining that a cell was zygosity assessable could maximize model accuracy and minimize sampling error, according to an example embodiment.

FIG. 15 outlines a method for generating, testing and using a logistic regression model for predicting LOH in individual cells and the prevalence of LOH in a population of cells, according to an example embodiment.

FIG. 16 shows how the zygosity score threshold and fraction of correct predictions correlate with the UMI coverage requirement, according to an example embodiment.

FIG. 17 shows an example of a logistic regression model instance for predicting whether cells were wild-type cells or LOH cells. By default, the probability cutoff was set to 0.5 (top panel) and the corresponding zygosity score (ΣMi/(ΣMi+ΣMa)) is defined as a zygosity score threshold; the zygosity score threshold in various model instances generated from different UMI coverage threshold (ΣMi+ΣMa) (bottom panel), according to an example embodiment.

FIG. 18 shows the LOH prevalence predicted for testing sample 1 (top panel) and testing samples 2 and 3 (bottom panel) using the indicated model instances, according to an example embodiment.

FIG. 19 illustrates the workflow of the systems and methods of the disclosure, according to an example embodiment.

FIG. 20 illustrates how the systems and methods of the disclosure can be used to determine whether LOH cells completely or partially lose heterozygosity in the LOH (top panel) and the effect of LOH on downstream gene expression (bottom panel), according to an example embodiment.

FIG. 21 illustrates the number of zygosity assessable cells that met a 1,000-cell minimum requirement for a testing sample 1 (TS1), a testing sample 2 (TS2), and a testing sample 3 (TS3).

FIG. 22 illustrates model instances that were trained and validated using cells that passed a UMI coverage requirement, according to an example embodiment.

### DETAILED DESCRIPTION

Machine learning methods are disclosed herein. The computer-implemented methods discussed herein may include additional, less, or alternate actions, including those discussed elsewhere herein. The methods may be implemented via one or more local or remote processors, transceivers, servers and/or via computer-executable instructions stored on non-transitory computer-readable media or medium.

Additionally, the computer systems discussed herein may include additional, less, or alternate functionality, including that discussed elsewhere herein. The computer systems discussed herein may include or be implemented via computer-executable instructions stored on non-transitory computer-readable media or medium.

A processor or a processing element may be trained using supervised or unsupervised machine learning, and the machine learning program may employ a neural network, which may be a convolutional neural network, a deep learning neural network, a reinforced or reinforcement learning module or program, or a combined learning module or program that learns in two or more fields or areas of interest. Machine learning may involve identifying and recognizing patterns in existing data in order to facilitate making predictions for subsequent data. Models may be created based upon example inputs in order to make valid and reliable predictions for novel inputs.

Additionally, or alternatively, the machine learning programs may be trained by inputting sample data sets or certain data into the programs, such as genetic data for populations of cells, and/or other data. The machine learning programs may utilize deep learning algorithms that may be primarily focused on pattern recognition, and may be trained after processing multiple examples. The machine learning programs may include Bayesian Program Learning (BPL), voice recognition and synthesis, image or object recognition, optical character recognition, and/or natural language processing—either individually or in combination. The machine learning programs may also include natural language processing, semantic analysis, automatic reasoning, and/or machine learning.

Supervised and unsupervised machine learning techniques may be used. In supervised machine learning, a processing element may be provided with example inputs and their associated outputs, and may seek to discover a general rule that maps inputs to outputs, so that when subsequent novel inputs are provided the processing element may, based upon the discovered rule, accurately predict the correct output. In unsupervised machine learning, the processing element may be required to find its own structure in unlabeled example inputs. In some embodiments, machine learning techniques may be used to extract data about a particular a prevalence of loss of heterozygosity (LOH) in a target population of cells from genetic data from reference populations of cells, and/or other data.

In some embodiments, the voice bots or chatbots discussed herein may be configured to utilize ML and/or AI techniques. For instance, the voice bot or chatbot may be an Artificial Intelligence (AI) bot (including generative AI bots). The AI bot may employ supervised or unsupervised machine learning techniques, which may be followed by, and/or used in conjunction with, reinforced or reinforcement learning techniques. The AI bot may employ the techniques utilized for ChatGPT.

As will be appreciated based upon the foregoing specification, the above-described embodiments of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), SD card, memory device and/or any transmitting/receiving medium, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are example only, and are thus not intended to limit in any way the definition and/or meaning of the term “processor.”

As used herein, the terms “software” and “firmware” are interchangeable, and include any computer program stored in memory for execution by a processor, including RAM memory, ROM memory, EPROM memory, EEPROM memory, and non-volatile RAM (NVRAM) memory. The above memory types are example only, and are thus not limiting as to the types of memory usable for storage of a computer program.

In some embodiments, a computer program is provided, and the program is embodied on a computer readable medium. In an exemplary embodiment, the system is executed on a single computer system, without requiring a connection to a server computer. In a further embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some embodiments, the system includes multiple components distributed among a plurality of computing devices. One or more components may be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes. The present embodiments may enhance the functionality and functioning of computers and/or computer systems.

The patent claims at the end of this document are not intended to be construed under 35 U.S.C. § 112(f) unless traditional means-plus-function language is expressly recited, such as “means for” or “step for” language being expressly recited in the claim(s).

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

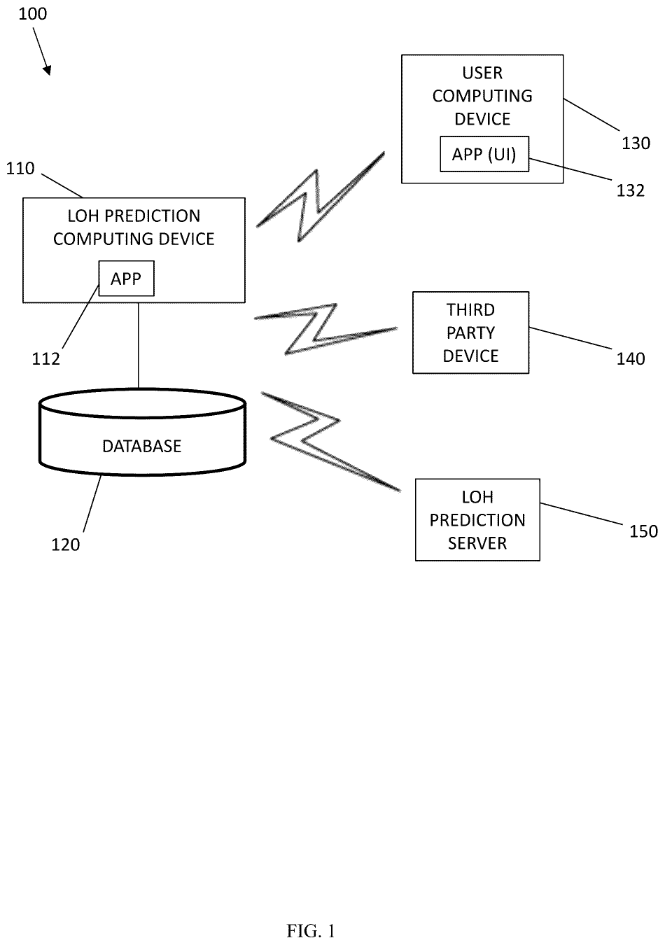

Exemplary computer systems for predicting loss of heterozygosity (LOH) are disclosed herein. For example, FIG. 1 depicts a schematic diagram of an example computer system 100. Computer system 100 is configured to predict LOH in a population of cells. In one example embodiment, computer system 100 may include and/or facilitate communication between a LOH prediction computing device 110 and one or more user computing devices 130 (which may also be referred to as “mobile devices”) and/or between LOH prediction computing device 110 and one or more of third-party devices 140 and/or LOH prediction servers 150.

LOH prediction computing device 110 may be implemented as a server computing device with artificial intelligence and deep learning functionality. Alternatively, LOH prediction computing device 110 (and/or user computing devices 130) may be implemented as any device capable of interconnecting to the Internet, including mobile computing device or “mobile device,” such as a smartphone, a “phablet,” or other web-connectable equipment or mobile devices (such as one or more local or remote processors, servers, transceivers, memory units, mobile devices, wearables, smart watches, smart contact lenses, smart glasses, augmented reality glasses, virtual reality headsets, mixed or extended reality glasses or headsets, voice or chat bots, artificial intelligence (AI) bots (including generative AI bots), and/or other electronic or electrical components, which may be in wired or wireless communication with one another).

LOH prediction computing device 110 may be in communication with one or more user computing devices 130, third party devices 140, and/or LOH prediction server 150, such as via wireless communication or data transmission over one or more radio frequency links or wireless communication channels. In the exemplary embodiment, components of computer system 100 may be communicatively coupled to the Internet through many interfaces including, but not limited to, at least one of a network, such as the Internet, a local area network (LAN), a wide area network (WAN), or an integrated services digital network (ISDN), a dial-up-connection, a digital subscriber line (DSL), a cellular telecommunications connection (e.g., a 3G, 4G, 5G, etc., connection), a cable modem, and a BLUETOOTH connection.

Computer system 100 also includes one or more database(s) 120 containing information on a variety of matters. For example, database 120 may include such information as genetic data and/or any other information used, received, and/or generated by computer system 100 and/or any component thereof, including such information as described herein. In one exemplary embodiment, database 120 may include a cloud storage device, such that information stored thereon may be securely stored but still accessed by one or more components of computer system 100, such as, for example, LOH prediction computing device 110, user computing devices 130, and/or LOH prediction servers 150. In some embodiments, database 120 may be stored on LOH prediction computing device 110. In an alternative embodiment, database 120 may be stored remotely from LOH prediction computing device 110 and may be non-centralized.

In some embodiments, user computing devices 130 may be computers that include a web browser or a software application to enable user computing devices 130 to access the functionality of LOH prediction computing device 110 using the Internet or a direct connection, such as a cellular network connection. User computing devices 130 may be any device capable of accessing the Internet including, but not limited to, a desktop computer, a mobile device (e.g., a laptop computer, a personal digital assistant (PDA), a cellular phone, a smartphone, a tablet, a phablet, netbook, notebook, smart watches or bracelets, smart glasses, wearable electronics, pagers, virtual reality headsets, augmented reality glasses, voice or chat bots, wearables, etc.), or other web-based connectable equipment.

User computing devices 130 may be used to access a data management app 112 maintained by LOH prediction computing device 110, for example, via a user interface 132 when data management app 112 is executed on user computing device 130. A user may use data management app 112 to provide inputs to LOH prediction computing device 110, view predictions generated by LOH prediction computing device 110, and perform other actions, including those described elsewhere herein.

Third party devices 140 may be computing devices associated with external sources of data. LOH prediction computing device 110 may request, receive, and/or otherwise access data from third party devices 140. Third party devices 140 may be any devices capable of interconnecting to the Internet, including a server computing device, a mobile computing device or “mobile device,” such as a smartphone, or other web-connectable equipment or mobile devices.

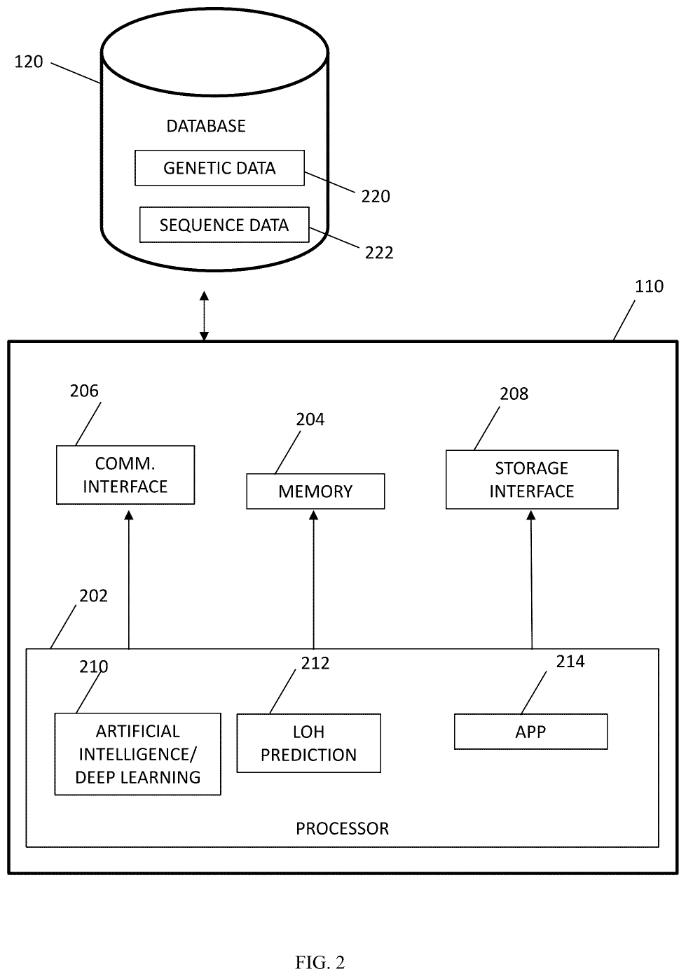

Exemplary user analytics computing devices are disclosed herein. For example, FIG. 2 depicts LOH prediction computing device 110 (as shown in FIG. 1), according to an embodiment. In some embodiments, LOH prediction computing device 110 may include a processor 202, a memory 204 (which may be similar to database 120, also shown in FIG. 1), a communication interface 206, and a storage interface 208. Processor 202 is configured to execute instructions, which may be stored in memory 204. Processor 202 includes one or more processing units (e.g., in a multi-core configuration) and may be configured to execute a plurality of modules.

In some embodiments, processor 202 is operable to execute an artificial intelligence/deep learning (AI/DL) module 210, an LOH prediction module 212, and a module 214 that maintains functionality for data management app 112 (shown in FIG. 1). Modules 210, 212, and 214 may include specialized instruction sets, and/or coprocessors. Database 120 and/or memory 204 may store any data and/or instructions necessary for modules 210, 212, and 214 to function as described herein. In the exemplary embodiment, database 120 may store genetic data 220, sequence data 222, and/or any other information used, received, and/or generated by LOH prediction computing device 110.

AI/DL module 210 may execute artificial intelligence and/or deep learning functionality on behalf of LOH prediction module 212. Specifically, AI/DL module 210 may include any rules, algorithms, training data sets/programs, and/or any other suitable data and/or executable instructions that enable LOH prediction computing device 110 employ artificial intelligence and/or deep learning to predict LOH in a population of cells.

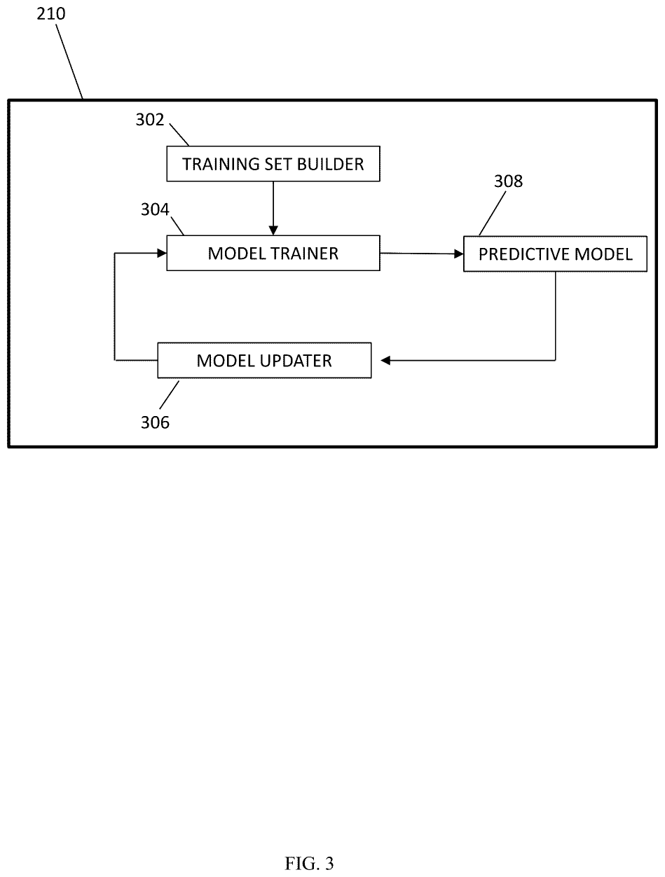

FIG. 3 depicts an AI/DL module 210 (as shown in FIG. 2), according to an embodiment. In some embodiments, AI/DL module 210 includes a training set builder module 302 programmed to submit one or more queries to database 120 (shown in FIGS. 1 and 2) to retrieve data and/or subsets of data, and to use those subsets to build training data sets for generating predictive model 308.

In example embodiments, training set builder module 302 is programmed to retrieve training data sets from the retrieved subsets of data. Each training data set corresponds to genetic data for a population of cells and the corresponding LOH for each cell of the population of cells. In some embodiments, training data corresponds to mapped identifiers for each cell of a population of cells and their corresponding LOH. Historical data may comprise previously determined LOH. Each training data set can include model input data along with result data representing a LOH. The model input data can represent factors that may be expected to, or unexpectedly be found during model training to, have some correlation with the LOH.

After training set builder module 302 generates training data sets, it passes the training data sets to model trainer module 304, which is programmed to apply the model input data fields of each training data set as inputs to one or more machine learning models. Each of the one or more machine learning models is programmed to produce, for each training data set, at least one output intended to correspond to, or “predict,” a value of the at least one result data field of the training data set. Machine learning can include various algorithms that may be used to train the model to identify and recognize patterns in existing data in order to facilitate making predictions for subsequent new input data.

Model trainer module 304 is programmed to compare, for each training data set, the at least one output of the model to the at least one result data field of the training data set, and apply a machine learning algorithm to adjust parameters of the model in order to reduce the difference or “error” between the at least one output and the corresponding at least one result data field. In this way, model trainer module 304 trains the machine learning model to accurately predict LOH for inputs. In other words, model trainer module 304 cycles the one or more machine learning models through the training data sets, causing adjustments in the model parameters, until the error between the at least one output and the LOH falls below a suitable threshold, and then uploads at least one trained machine learning model to predictive model module 308 for application to new structure data (e.g., a primary amino acid sequence).

In some embodiments, the one or more machine learning models may include one or more neural networks, such as a convolutional neural network, a deep learning neural network, or the like. The neural network may have one or more layers of nodes, and the model parameters adjusted during training may be respective weight values applied to one or more inputs to each node to produce a node output. In other words, the nodes in each layer may receive one or more inputs and apply a weight to each input to generate a node output. The node inputs to the first layer may correspond to the model input data fields, and the node outputs of the final layer may correspond to the at least one output of the model, intended to predict the at least one result data field. One or more intermediate layers of nodes may be connected between the nodes of the first layer and the nodes of the final layer. As model trainer module 304 cycles through the training data sets, model trainer module 304 applies a suitable backpropagation algorithm to adjust the weights in each node layer to minimize the error between the at least one output and the corresponding result data field. In this fashion, the machine learning model is trained to produce one or more outputs which reliably predicts LOH for a population of cells. Alternatively, the machine learning model has any suitable structure. In some embodiments, model trainer module 304 provides an advantage by automatically discovering and properly weighting complex, second- or third-order, and/or otherwise nonlinear interconnections between the model input data fields and the at least one output. Absent the machine learning model, such connections are unexpected and/or undiscoverable by human analysts.

Additionally, or alternatively, the one or more machine learning models may include one or more multilayer perceptron (MLP) classifiers. A MLP classifier may comprise input and output layers, and one or more hidden layers with many neurons stacked together.

Additionally, or alternatively, the one or more machine learning models may include one or more support vector machines (SVMs). SVMs are supervised learning models with associated learning algorithms that analyze data for classification and regression analysis. More particularly, a SVM constructs a hyperplane or set of hyperplanes in a high or infinite-dimensional space, which can be used for classification, regression, or other tasks like outliers detection.

In some embodiments, predictive model module 308 compares the known LOH for the population of cells with the output from the trained model, and routes the comparison result to a model updater module 306 of AI/DL module 210. Model updater module 306 is programmed to use the comparison results to enable updating or “re-training” of the at least one machine learning model to improve performance. The retrained machine learning model may be periodically re-uploaded to predictive model module 308.

In some embodiments, model trainer module 304 may update the training dataset by creating one or more new historical records which includes new data and re-training the operator model using the updated training dataset, further improving the accuracy of the operator model.

LOH prediction module 212 may employ AI/DL module 210 to use the trained model to predict LOH for a population of cells. More particularly, LOH prediction module 212 may use the output from the trained model to predict a LOH for a population of cells. The predicted LOH and other data may be viewable via data management app 112.

App module 214 is configured to facilitate maintaining data management app 112 and providing the functionality thereof to users. App module 214 may store instructions that enable the download and/or execution of data management app 112 at user computing devices 130. App module 214 may store instructions regarding user interfaces, controls, commands, settings, and the like, and may format data into a format suitable for transmitting to user computing devices 130 for display thereof.

In some embodiments, processor 202 is operatively coupled to communication interface 206 such that LOH prediction computing device 110 is capable of communicating with remote device(s) such as user computing devices 130, third party devices 140, and/or LOH prediction servers 150 (all shown in FIG. 1) over a wired or wireless connection. For example, communication interface 206 may receive genetic data and the like, from user computing devices 130, and/or third-party devices 140. Communication interface 206 may include, for example, a wired or wireless network adapter and/or a wireless data transceiver for use with a mobile telecommunications network.

Processor 202 may also be operatively coupled to database 120 (and/or any other storage device) via storage interface 208. Database 120 may be any computer-operated hardware suitable for storing and/or retrieving data. In some embodiments, database 120 may be integrated in LOH prediction computing device 110. For example, LOH prediction computing device 110 may include one or more hard disk drives as database 120. In other embodiments, database 120 is external to LOH prediction computing device 110 and is accessed by a plurality of computer devices. For example, database 120 may include a storage area network (SAN), a network attached storage (NAS) system, multiple storage units such as hard disks and/or solid-state disks in a redundant array of inexpensive disks (RAID) configuration, cloud storage devices, and/or any other suitable storage device.

Storage interface 208 may be any component capable of providing processor 202 with access to database 120. Storage interface 208 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 202 with access to database 120.

Processor 202 may execute computer-executable instructions for implementing aspects of the disclosure. In some embodiments, processor 202 may be transformed into a special purpose microprocessor by executing computer-executable instructions or by otherwise being programmed. For example, processor 202 may be programmed with instructions.

Memory 204 may include, but is not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are example only, and are thus not limiting as to the types of memory usable for storage of a computer program.

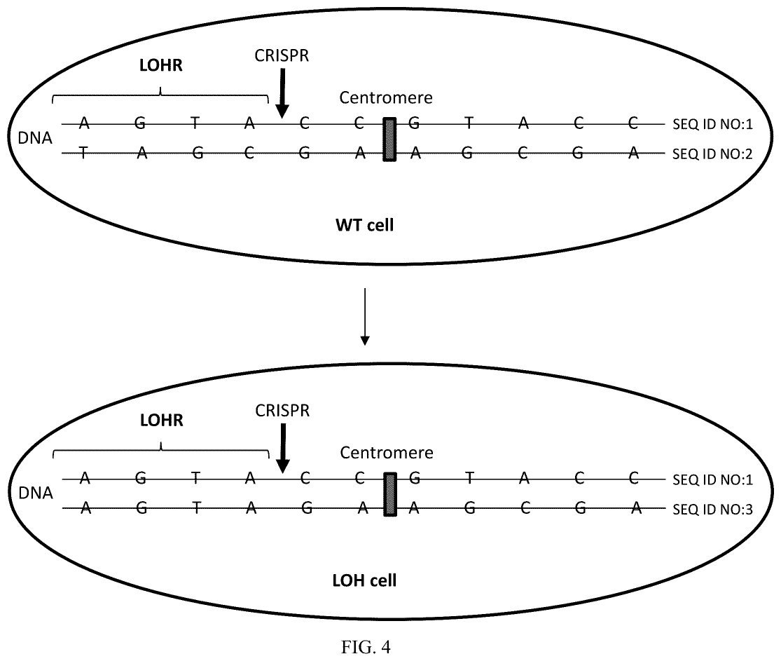

FIG. 4 shows a chromosome region where zygosity is of interest (LOHR), according to an example embodiment. A LOHR in CRISPR editing-induced copy-neutral loss of heterozygosity (LOH) is the DNA segment from a CRISPR editing site to the end of the telomere on the same chromosome arm. In different cells, LOHR can be wild-type (WT) or LOH. The prevalence of CRISPR-induced copy-neutral LOH is about 5% to about 16%; however, a need exists for a high-throughput method that can accurately assess the prevalence of LOH in a CRISPR-treated cell population.

Generating a model for predicting LOH can be complicated. A model for predicting LOH based on one data set which includes a first set of cell type, CRISPR editing site, and 10x Genomics data, may not be applicable to another data set comprised of a different set of cell type, CRISPR editing site, and 10x Genomics data. In some instances, pure LOH cells may not be available to train a model for predicting individual cell LOH.

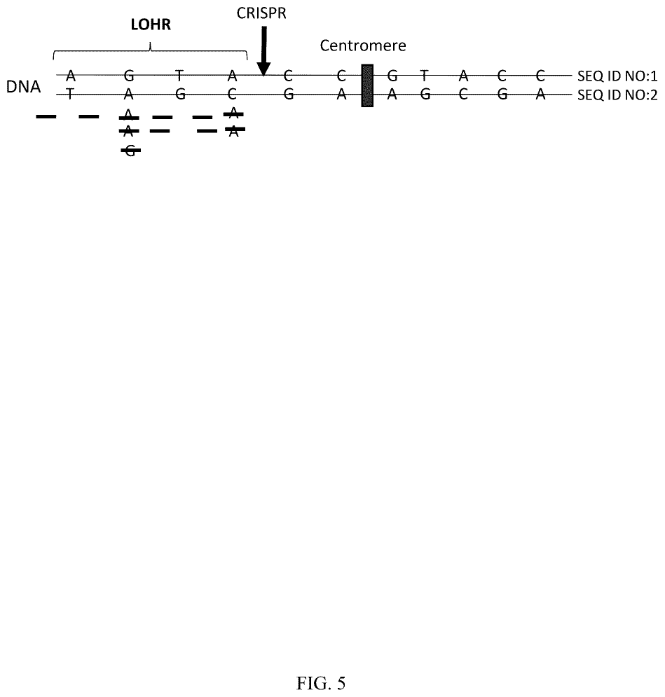

Additionally, the coverage of heterozygous variant positions in a LOHR covered by unique molecular identifiers (UMIs) is usually low. FIG. 5 illustrates a chromosome region of a strand of wild-type DNA where zygosity is of interest (LOHR) and further shows how few UMIs that derive from single-cell RNA sequencing may map to the heterozygous positions in LOHRs. It has been found that only a fraction of the about 4,000 to about 12,000 mRNA molecules sequenced using single-cell RNA sequencing may map to a LOHR, and the sequence length of each sequenced mRNA molecule may be about 80 nucleotides, while single nucleotide polymorphisms comprise about 0.1% of the human genome. Therefore, single-cell RNA sequencing can present various challenges, including accounting for sequencing errors. For example, determining the minimum UMI coverage requirement of a single variant position for inclusion in zygosity assessment can be difficult. Furthermore, the overall low UMI coverage may prevent assessing the zygosity of some cells and complicate finding the UMI coverage threshold. Producing one LOHR zygosity measurement for a cell from multiple variant positions can also be challenging.

The systems and methods of the present disclosure can detect the prevalence of LOH in a cell population by inferring the zygosity of the LOHR with over 99% sensitivity and specificity at the single-cell level. In various aspects, the systems and methods utilize a single-cell RNA sequencing-based, high throughput process that does not require cell cloning. The systems and methods can also be employed independent of the cell type, the CRISPR editing site, and the 10x genomics single-cell RNA sequencing platform. The systems and methods use UMIs instead of conventional read count-based zygosity calling. The systems and methods can utilize machine learning-based LOH predictions and mLOH cell data for model training derived from wild-type cells instead of purified LOH cells. Prediction model instance selection can be made by balancing the model performance on individual cell prediction and minimizing sampling error for the estimation of the LOH prevalence in a cell population.

In some aspects, the present disclosure provides systems and methods wherein a variant position is included in HetRef if it is covered by at least about 20 DNA sequencing reads and/or each of the two variants cover more than about 20% of the DNA sequencing reads.

In other aspects, HomRef can be generated by establishing a set of homozygous positions that are a certain nucleotide distance away from the variant positions registered in HetRef2. A UMI can be generated from the single-cell RNA sequencing cells from the second population of cells (WT) mapped to HetRef2 or HomRef, which can be used as training and validation datasets of WT or mLOH cells, respectively, in logistic model training to predict loss of heterozygosity. Prediction models using coverage and zygosity score can be used to predict loss of heterozygosity. In various aspects, suitable prediction models may include logistic regression models, tree-based methods, neural networks and support vector machines, k-nearest neighbor or other traditional statistical methods. In one aspect, a logistic regression model is used.

Unless described otherwise, all technical and scientific terms used herein have the same meaning as commonly understood by one of ordinary skill in the art to which this disclosure belongs. Although any methods and materials similar or equivalent to those described herein can be used in the practice or testing, particular methods and materials are now described.

The term “a” refers to “at least one” and the terms “about” and “approximately” refer to a permitted standard variation as would be understood by those of ordinary skill in the art, and where ranges are provided, endpoints are included. As used herein, the terms “include,” “includes,” and “including” are meant to be non-limiting and refer to “comprise,” “comprises,” and “comprising”, respectively.

The term “DNA segment” refers to a length of genomic DNA that may be delineated by its ability to be sequenced using DNA probes.

The term “bulk DNA sequencing” or “bulk DNAseq” refers to sequencing DNA derived from a population of cells.

The term “genome” refers to the DNA or RNA genetic material present within a cell, including the chromosomal/nuclear genome of a cell, as well as any mitochondrial and/or plasmid genome. The nuclear genome can include protein-coding genes, non-coding genes, other functional regions, including non-coding DNA or RNA, and junk DNA or RNA, if present. In some embodiments, the genome is comprised in a cell. In some embodiments, the genome is comprised in a cell from an established cell line (e.g., a 293T cell), or a primary cell cultured ex vivo (e.g., cells obtained from a subject and grown in culture). In some embodiments, the genome is comprised in a hematologic cell (e.g., hematopoietic stem cell, leukocyte, or thrombocyte), or a cell from a solid tissue, such as a liver cell, a kidney cell, a lung cell, a heart cell, a bone cell, a skin cell, a brain cell, or any other cell found in a subject. In some embodiments, the genome or the cell comprising the genome is in a subject. Subjects comprising the genomes of the present disclosure include, but are not limited to, humans and/or other primates; mammals, including, but not limited to, cattle, pigs, horses, sheep, cats, dogs, mice, and/or rats; and/or birds, including commercially relevant birds such as chickens, ducks, geese, and/or turkeys.

The term “protein-coding gene” refers to mean sequences of nucleic acid molecules (RNA or DNA molecules) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

The term “non-coding” refers to mean nucleotide sequences in a cell that do not encode protein sequences. Non-coding DNA can be transcribed into functional non-coding RNA molecules, including transfer RNAs, microRNAs, piRNAs, ribosomal RNAs and regulatory RNAs. Functional regions of non-coding DNA include regulatory nucleotide sequences that control gene expression, scaffold attachment regions, origins of DNA replication, centromeres and telomeres. Non-coding DNA regions, including introns, pseudogenes, intergenic DNA, and fragments of transposons and viruses, can appear to be mostly or entirely nonfunctional.

The term “single-cell RNA sequencing” or “scRNAseq” refers to a method for the detection and quantitative analysis of messenger RNA molecules within an individual cell.

The term “chromosome coordinate” refers to a position, or a range of positions within a reference genome on a specific chromosome, in the latter case, including a starting position and ending position.

The term “nucleotide composition” refers to the DNA or RNA nucleotide(s) detected in a chromosome coordinate.

The term “genetic data” refers to information regarding the genetic material present in a cell, and may include the sequence of nucleotide bases that make up DNA or RNA, as well as information about the arrangement and expression of genes.

The term “loss of heterozygosity” or “LOH” refers to the replacement of one DNA allele with the other in a chromosomal region, within a diploid organism.

The term “mock loss of heterozygosity” or “mLOH” refers to the loss of heterozygosity single-cell RNA sequencing data that are digitally generated from wild-type cells by calculating the coverage and zygosity score of the homozygous positions in the homozygous reference using Equation 1 and Equation 2, respectively.

The term “CRISPR” refers to an enzyme system comprising a guide RNA sequence comprising a nucleotide sequence that is complementary or substantially complementary to a target polynucleotide region and a protein having nuclease activity. CRISPR-Cas systems include type I, type II, or type III CRISPR-Cas systems and their derivatives CRISPR-Cas systems, and further include engineered and/or programmed nuclease systems derived from naturally occurring CRISPR-Cas systems. The CRISPR-Cas system can comprise an engineered and/or mutated Cas protein. The CRISPR-Cas system can include engineered and/or programmed guide RNAs.

As used herein, the term “guide RNA” refers to an RNA that includes a sequence that is complementary or substantially complementary to a region of a target DNA sequence. The guide RNA can include a nucleotide sequence that is not complementary or substantially complementary to a region of the target DNA sequence. The guide RNA can be a crRNA or a derivative thereof, such as crRNA: tracrRNA chimeras.

As used herein, the term “nuclease” refers to an enzyme capable of cleaving phosphodiester bonds between nucleotide subunits of a nucleic acid. The term “endonuclease” refers to an enzyme capable of cleaving phosphodiester bonds within a polynucleotide strand. The term “nickase” refers to an endonuclease that cleaves only a single strand of a DNA duplex. The term “Cas 9 nickase” refers to a nickase derived from Cas9protein, typically by inactivating one nuclease domain of the Cas9 protein.

The term “loss of heterozygosity region” or “LOHR” refers to the chromosome region where zygosity is of interest. Regarding CRISPR-induced copy-neutral LOH, a LOHR can be the DNA segment from a CRISPR editing site to the end of the telomere on the same chromosome arm.

The term “major allele” or “Ma” refers to the allele including the more frequently detected nucleotide or higher UMI coverage in scRNAseq at a variant position.

The term “minor allele” or “Mi” refers to the allele including the less frequently detected nucleotide or lower UMI coverage in scRNAseq at a variant position.

The term “wild-type” or “wt” refers to a cell for which multiple copies of the same chromosome have unique nucleotide sequences. For example, in a diploid cell, both copies of a chromosome have unique nucleotide sequences. The term “wild-type” or “wt” also refers to a non-genome edited, non-mutated cell and is thus useful as a reference for a comparison with a genome edited cell. A “Unique Molecular Identifier” or “UMI” is a barcode used in single cell RNA sequencing that refers to a particular mRNA molecule being sequenced. The number of UMI mapped to a particular position is the same as the number of mRNA molecules sequenced covering the same position. The UMI barcode is added to the nucleic acids in the nucleic acid library prior to its sequencing. The particular UMI serves to identify sequencing reads stemming for example from the same specific cell.

The term “coverage” refers to the number of UMIs covering a particular chromosome coordinate in HetRef2 or HomRef or in the case for the evaluation of LOHR zygosity of a cell, it is the sum of the number of UMIs covering all chromosome coordinates registered in the LOHR of HetRef2 or HomRef in a cell. Equation 1 can be used, by the processor 202, to calculate the UMI coverage of LOHR in a cell.

Coverage=ΣMₐΣ+ΣMᵢ Equation 1

where ΣMₐ and ΣMᵢ, are the sum of the number of UMIs covering major and minor alleles, respectively, across all LOHR variant positions registered in HetRef2 or HomRef.

The term “zygosity score” refers to the ratio of the sum of the number of UMIs covering the minor allele (Mi) to the total coverage, which is calculated by Equation 1. Equation 2 can be used, by the processor 202, to calculate the zygosity score of a cell.

$$Zygosity Score = \frac{Σ {M}_{i}}{Σ {M}_{a} + Σ {M}_{i}} \qquad Equation 2$$

The term “zygosity score threshold” refers to a zygosity score from a model in which any zygosity score greater than or equal to the threshold is predicted as heterozygous, any zygosity score less than the threshold is predicted as homozygous or LOH.

The term “reads” refers to sequences of nucleotides corresponding to all or part of a molecule comprising nucleotides that are generated using nucleic acid sequencing technologies, including, for example, DNA sequencing, RNA sequencing, single-cell DNA sequencing and single-cell RNA sequencing.

The term “heterozygous variant positions having imbalanced allelic expression” or “allelic expression imbalanced heterozygous variants” refers to a heterozygous variant position that is covered by at least about 100 UMIs and where at least one of two alleles is represented in at least about 80% of all UMIs, which can be detected using single-cell RNA sequencing data of wild-type cells to generate pseudo bulk gene expression data. These variants can also be detected using real bulk RNA sequencing.

The term “10x genomics” refers to single-cell RNA-sequencing technology in which the Chromium Single Cell 3′ solution allows analysis of transcriptomes on a cell-by-cell basis using microfluidic partitioning to capture single cells and prepare barcoded, next-generation sequencing (NGS) cDNA libraries. Single cells, reverse transcription (RT) reagents, gel beads including barcoded oligonucleotides and oil are combined on a microfluidic chip to form reaction vesicles called gel beads in emulsion, or GEMs. GEMs are formed in parallel within the microfluidic channels of the chip, allowing a user to process about hundreds to about tens-of-thousands of single cells in a single 7-minute Chromium instrument run. Cells are loaded at a limiting dilution to maximize the number of GEMs including a single cell and ensure a low doublet rate, while maintaining a high cell recovery rate of up to about 65%.

Each functional GEM contains a single cell, a single gel bead, and reverse transcription reagents. Within each gel bead in emulsion reaction vesicle, a single cell is lysed, the gel bead is dissolved to free the identically barcoded reverse transcription oligonucleotides into solution, and reverse transcription of polyadenylated mRNA occurs. As a result, all cDNAs from a single cell will have the same barcode, allowing the sequencing reads to be mapped back to their original single cells of origin. The preparation of next-generation sequencing libraries from these barcoded cDNAs is then carried out in a highly efficient bulk reaction.

As used herein, a “heterozygous variant reference” or “HetRef” refers to a first heterozygous variant reference including heterozygous variant positions, including the chromosome coordinates and nucleotide composition of the heterozygous variant, which can be identified using whole genome bulk DNA sequencing of wild-type cells for LOHR. HetRef may not contain haplotype information. A HetRef can be generated using whole genome bulk DNA sequencing of wild-type cells. A chromosome coordinate can be determined to be a heterozygous variant position if the chromosome coordinate is covered by at least about 20 reads and the associated allele is represented in between about 20% and about 80% of reads produced using whole genome bulk DNA sequencing.

As used herein, a “second heterozygous variant reference” or “HetRef2” refers to a heterozygous variant reference that is created by removing heterozygous variant positions from HetRef that may have allelic expression imbalance.

Impurity information from all eligible heterozygous variant positions of HetRef2 in a LOHR can be compiled, by the processor 202, to form an artificial composite variant using Equation 1 (e.g., the sum of the number of UMIs that cover a more frequently detected nucleotide at each variant position and the number of UMIs that cover a less frequently detected nucleotide at each variant position) to determine a UMI coverage. Heterozygous variant positions of HetRef2 with coverage by at least about 4 UMIs at each position may be included. Equation 2 can be used, by the processor 202, to determine the zygosity score, which is the same as coverage-weighted average percentage across all eligible variant positions, and the zygosity score can be used, by the processor 202, to build the simple logistic regression model.

In an example embodiment, zygosity can be assessed, by the processor 202, using a probability-based method. Assuming both alleles are transcribed at the same rate, Equation 3 can be used, by the processor 202, to calculate the probability of observing that r or more out of n UMIs covering a chromosome coordinate that is a presumably heterozygous variant position contain the same nucleotide,

$$p = {\sum }_{i = r}^{n} \frac{n ! / [ i ! * ( n - i ) ! ]}{{2}^{n - 1}} \{ r \geq \frac{1}{2} n \} \qquad Equation 3$$

where n is the number of UMIs covering the chromosome coordinate that is a presumably variant position and r is the number of UMIs covering the more observed nucleotide at the variant position. For example, the chance of observing 9 or more UMIs with the same nucleotide (r=9) at a chromosome coordinate that is a heterozygous variant position with a coverage of 10 UMIs (n=10) is 2.1484% if both alleles are transcribed at the same rate. Equation 4 can be used, by the processor 202, to calculate the probability of observing that all UMIs covering a chromosome coordinate that is a heterozygous variant position contain the same nucleotide,

$$\{ n = r \} \to p = \frac{1}{{2}^{n - 1}} \qquad Equation 4$$

and Table 1 shows the probability of observing that all UMIs covering a chromosome coordinate that is a heterozygous variant position consisting of adenine (A) and thymine (T) contain the same nucleotide of A or T for n=r and r=2, 3, 4, 5 or 6.

TABLE 1

| Unique | | |
|---|---|---|
| Molecular | | |
| Identifier | | Probability |
| Coverage of | | Only Same |
| Heterozygous | Equally Possible | Nucleotide |
| A/T | Outcomes | Observed |
| 2 | AA, AT, TA, TT | 50.000% |
| 3 | AAA, AAT, ATA, | 25.000% |
| | ATT, TAA, TAT, | |
| | TTA, TTT | |
| 4 | AAAA, AAAT, AATA, | 12.500% |
| | ATAA, TAAA, AATT, | |
| | ATTA, TTAA, TAAT, | |
| | ATAT, TATA, ATTT, | |
| | TTTA, TTAT, TATT, | |
| | TTTT | |
| 5 | AAAAA, . . . , TTTTT | 6.250% |
| 6 | AAAAAA, . . . , TTTTTT | 3.125% |

The probability-based method is sensitive to coverage (n). When the coverage is low, for example, lower than 4 UMIs, even if it is a true heterozygous variant position, the chance to observe all the UMIs that have the same nucleotide is high (≥25%).

Alternatively, nucleotide zygosity can also be assessed, by the processor 202, using impurity-based methods, including, for example, the Gini Index, Entropy and Percentage. Equation 5 can be used, by the processor 202, to calculate the Gini Index,

Gini Index: 1−p²−(1−p)² Equation 5

Where p is the UMI proportion of one of the two nucleotides covering a variant position. Equation 6 can be used, by the processor 202, to calculate the Entropy,

Entropy: −p*log₂ p−(1−p)*log₂(1−p) Equation 6

where p is the UMI proportion of one of the two nucleotides covering a variant position. Equation 7 can be used, by the processor 202, to calculate the Percentage,

Percentage: min(p,1−p) Equation 7

where p is the proportion of two nucleotides covering a variant position. For p≤ 0.5, all three impurity calculation methods are monotonic functions, which means the bigger the p value, the higher the impurity assessment value. If a rank order-based method (e.g., logistic regression or decision tree) is used, by the processor 202, for a later LOH prediction model, the prediction accuracy will be identical by using any one of the Gini Index, Entropy or Percentage impurity-based methods.

As used herein, “homozygous reference” or “HomRef” refers to a reference including the homozygous coordinates within ±100 nucleotides of each heterozygous variant coordinate registered in HetRef2. In some embodiments, a HomRef is used to generate mLOH cells by calculating the zygosity score and coverage of the HomRef positions. In some embodiments, a HomRef is used to generate mLOH cells from wild-type cells for training a LOH model. A mLOH cell can be digitally generated from a wild-type cell and its zygosity score scored can be calculated by using UMIs covering homozygous variant reference positions in a LOHR of a wild-type cell. The zygosity scores of mLOH cells can be used together with those of wild-type cells to train a LOH prediction model.

Impurity information from all eligible heterozygous variant positions of HetRef2 in the LOHR of wild-type cells and other cells can be compiled, by the processor 202, to form an artificial composite variant with UMI coverage equal to the sum of the number of UMIs of the more frequently detected nucleotide in each variant position, or EA/a, and the number of UMIs of the less frequently detected nucleotide in each variant position, or EM, as seen in Equation 1, before determining the minimum coverage to deem a cell zygosity accessible, according to an example embodiment.

The minimum UMI coverage requirement should be applied, by the processor 202, to selecting wild-type cells and other cells of interest for model training and model independent validation, as well as cells from testing samples for LOH prevalence detection. Although a chromosome coordinate assessed may be homozygous, the zygosity score may not equal 0 in LOH or mLOH cells for various reasons that may include but are not limited to sequencing error, utilized homozygous positions registered in HomRef not being truly homozygous in mLOH cells and heterozygosity in the LOHR not being completely lost in LOH cells. The minimum UMI coverage requirement is different from the UMI coverage threshold used for the eligibility of a variant position to be included in a cell.

Equation 8 can be used, by the processor 202, to calculate the necessary number of cells to be assessed for zygosity to establish the LOH prevalence in a cell population,

$$n = \frac{{Z}^{2} p ( 1 - p )}{{d}^{2}} \qquad Equation 8$$

where p is the assumed prevalence, d is the precision (d) and Z is the Z-value for the desired confidence level.

In various aspects of the systems and methods of the disclosure, a target population of cells may comprise cells edited using a genome-editing tool, such as CRISPR. In some aspects, the target population of cells may include cells that have lost heterozygosity by one or more genome-editing procedures. In various aspects, a reference population of cells may include cells untreated with CRISPR, i.e., WT cells, cells having the same DNA genotype as WT cells, and/or cells having heterozygosity in a DNA segment of interest that can be used to generate HetRef.

In various aspects, reference data may comprise HetRef, HetRef2, and/or HomRef.

The present disclosure will be more fully understood by reference to the following Examples, though the Examples should not be construed as limiting the scope of the disclosure.

### EXAMPLES

### Methods

Three cell populations were sequenced. All three cell populations were from the same subject. The first cell population was used to perform bulk DNA sequencing to obtain the heterozygous reference (HetRef). The first population of cells can be any haplotype cells from the subject. The second (training sample) and third (testing sample) were used for scRNAseq. The second and third cell populations were the same cell type(s). The second population did not involve any CRISPR procedure, therefore the second population was considered to be wild type cells, while the third population did involve a CRISPR procedure. In the case of studying LOH in cancer, the second population is a non-tumor sample, while the third population is a tumor sample of the same tissue/organ as the non-tumor sample. The scRNA seq results of the second population were used: i) to identify the positions in HetRef that the two alleles have imbalanced expression, which were removed from HetRef to generate HetRef2; HomRef (a set of DNA coordinates that are a certain number of nucleotides away from the positions registered in HetRef2) was then generated from HetRef2; ii) mapped UMIs to HetRef2 to generate WT cell profiles (both coverage and zygosity scores); iii) mapped UMIs to HomRef to generate mLOH cell profiles (both coverage and zygosity scores); iv) and trained the zygosity prediction model (to predict the zygosity of individual cells) using the WT and mLOH zygosity scores (based on the different coverage requirement, multiple prediction model instances can be generated). The UMIs generated from the scRNAseq of the third population were mapped to HetRef2 to generate a profile for each cell (both coverage and zygosity scores) and were used to: i) select the prediction model instance generated form the second population by providing the number of cells in the third population that meet the coverage requirement of each prediction model instance (ideally, a model instance is selected that has good prediction accuracy and also has a coverage requirement that allows a enough qualifying cells in the third population to be predicted to minimize the population sampling error); ii) using the selected model instance to qualify each cell in the third population (the qualification is based on coverage) for predicted zygosity using its zygosity score; and iii) the percentage of LOH among the qualifying cells in the third population was calculated.

All cells were derived from human induced pluripotent stem cells (iPSC). Clonal loss of heterozygosity cells were edited using CRISPR genome editing targeted to coordinate 31,191,431 (C->T) on chromosome 16 based on GRCh38/hg38 assembly. Initially, the zygosity score of the loss of heterozygosity region was determined for each wild-type cell and loss of heterozygosity cell. A simple logistic regression model that transformed the zygosity score of a cell into genotype probability was then trained and cross-validated using 70% of the zygosity score data from wild-type cells and loss of heterozygosity cells. Next, the sensitivity, specificity and accuracy of the simple logistic regression model was independently validated using 30% of the zygosity score data from wild-type cells and loss of heterozygosity cells. Finally, the simple logistic regression model was used to assess the zygosity score of individual cells in a testing sample to find the loss of heterozygosity prevalence.

Wild-type cells were assayed using whole genome bulk DNA sequencing. A chromosome coordinate was determined to be heterozygous variant position if the chromosome coordinate was covered by at least about 20 reads and the proportion of one allele is between about 20% and about 80% of reads in the whole genome bulk DNA sequencing dataset. Heterozygosity for each coordinate can also be established by an existing algorithm developed for DNA sequencing data. Bulk DNA sequencing identified 37,902 variant positions on chromosome 16 of wild-type cells and 15,465 variant positions identified were in the loss of heterozygosity region on chromosome 16, according to an example embodiment.

Wild-type cells, clonal loss of heterozygosity cells and three testing samples that contained wild-type cells and 3%, 10% or 30% loss of heterozygosity cells were assayed using single-cell RNA sequencing. Table 2 shows the number of wild-type cells, loss of heterozygosity cells and cells from the three testing samples that were assayed using single-cell RNA sequencing.

TABLE 2

| Cell Population Composition | Cell Number |
|---|---|
| Wild Type | 7,238 |
| Loss of Heterozygosity | 7,767 |
| 3% Loss of Heterozygosity | 7,743 |
| 10% Loss of Heterozygosity | 7,886 |
| 30% Loss of Heterozygosity | 7,927 |

A first heterozygous variant reference including the chromosome coordinate and nucleotide composition of the 37,902 heterozygous variant positions identified on chromosome 16 of wild-type cells was generated and 15,465 variant positions identified were in the loss of heterozygosity region on chromosome 16. To establish the feasibility of using RNA sequencing data instead of DNA sequencing data for zygosity calling, single cell RNA sequencing data generated from a second or third cell population were treated as pseudo bulk by ignoring the cell identifying information in the data and using UMI instead of reads as coverage for the zygosity calling. A chromosome coordinate was determined to be heterozygous variant position if the chromosome coordinate was covered by at least about 20 unique molecular identifiers, the nucleotide composition obtained using single-cell RNA sequencing was the same as the nucleotide composition identified using whole genome bulk DNA sequencing, and the proportion of one allele was represented in between about 20% and about 80% of all unique molecular identifiers.

### Results

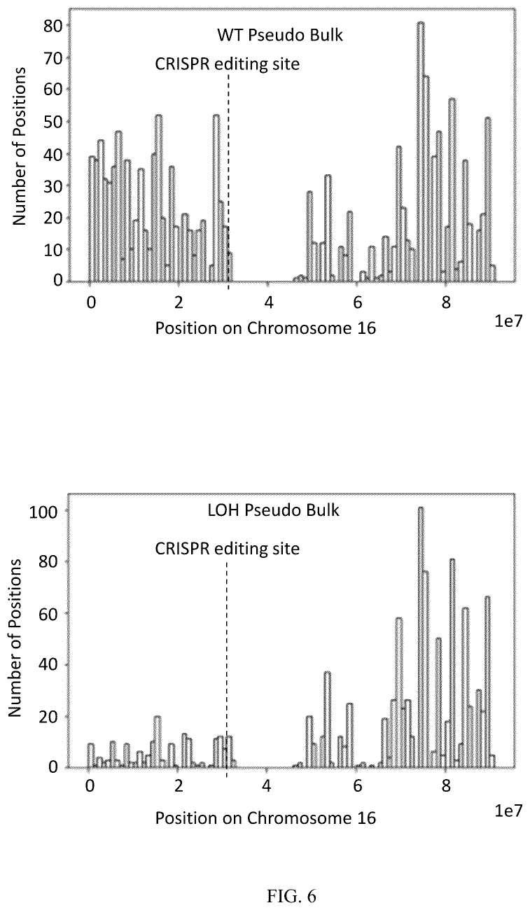

FIG. 6 shows the distribution of heterozygous variant positions identified on chromosome 16 of wild-type cells (top panel) and loss of heterozygosity cells (bottom panel) using single-cell RNA sequencing and the first heterozygous variant reference as a reference, according to an example embodiment. There were significantly more heterozygous positions in the LOHR in the wild type cells than in LOH cells, which confirms the potential of using RNA sequencing results generated from single cell experiments to infer DNA zygosity. Possible reasons as to why heterozygous variant positions were observed in the LOH cells include, for example: (i) LOH cells didn't lose heterozygosity in all heterozygous variant positions in LOHR, or (ii) a sequencing error in single cell RNA sequencing.

Potential allelic expression imbalance among the first heterozygous variant reference positions were identified using the first heterozygous variant reference as a reference. A heterozygous variant position in the first heterozygous variant reference was determined to potentially have allelic expression imbalance if the heterozygous variant position was covered by at least about 100 unique molecular identifiers in the pseudo bulk RNA sequencing data of the wild type cells (second population cells) and the proportion of one of the two alleles was represented in at least about 80% of all unique molecular identifiers. A total of 456 heterozygous variant positions in the first heterozygous variant reference were found to be allelic expression imbalanced, 276 of which were in the loss of heterozygosity region and 180 of which were outside the loss of heterozygosity region.

A second heterozygous variant reference was generated by removing the heterozygous variant positions in the first heterozygous variant reference that may have allelic expression imbalance. A total of 37,446 heterozygous variant positions were identified on chromosome 16 of wild-type cells in the second heterozygous variant reference, 15,189 of which were in the loss of heterozygosity region and 22,257 of which were outside the loss of heterozygosity region.

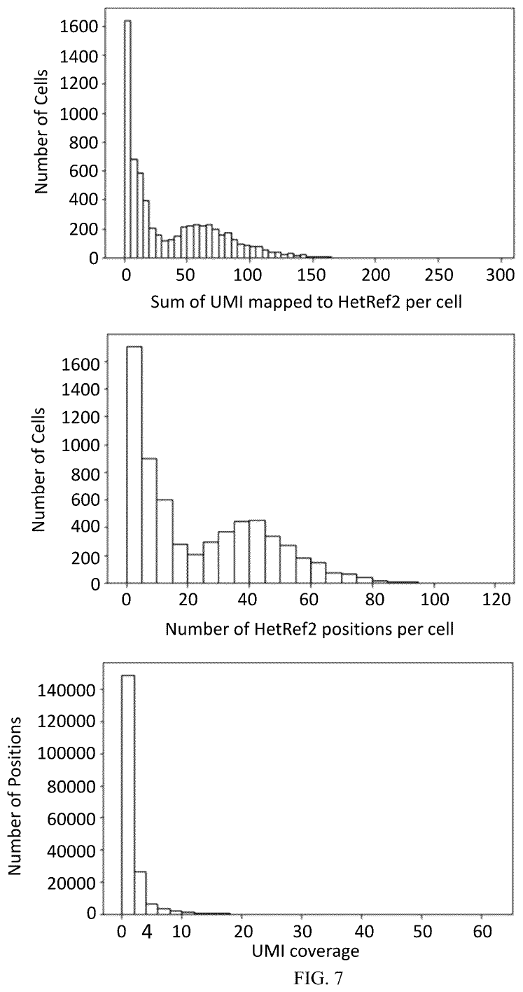

FIG. 7 shows that the unique molecular identifier coverage of the heterozygous variant positions of the second heterozygous variant reference was low in the loss of heterozygosity region on chromosome 16 of wild-type cells, according to an example embodiment. The top panel of FIG. 7 shows the total number of unique molecular identifiers covering the heterozygous variant positions of the second heterozygous variant reference in the loss of heterozygosity region of each wild-type cell was low, according to an example embodiment. The middle panel of FIG. 7 shows the number of heterozygous variant position of the second heterozygous variant reference covered by unique molecular identifiers in the loss of heterozygosity region of each wild-type cell was low, according to an example embodiment. The bottom panel of FIG. 7 shows the coverage of heterozygous variant positions of the second heterozygous variant reference by unique molecular identifiers in the loss of heterozygosity region of all wild-type cells was low, according to an example embodiment.

Equation 3 is a probability test that was used to understand the minimum unique molecular identifier coverage required for a heterozygous variant position in the second heterozygous variant reference to be included in the zygosity assessment of a cell. The null hypothesis for Equation 3 was that the variant position being assessed was heterozygous. Specifically, Equation 3 calculated the probability of observing that r or more out of n unique molecular identifiers that covered a variant position contained the same nucleotide given the hypothesis that the position being assessed was heterozygous and both alleles were transcribed at the same rate.

For example, the chance of observing 3 or more unique molecular identifiers with the same nucleotide (r=3) at a heterozygous variant position with a coverage of 4 unique molecular identifiers (n=4) is 50% if both alleles are transcribed at the same rate. Equation 4 calculated the probability of observing that all unique molecular identifiers that covered a variant position contained the same nucleotide if both alleles were transcribed at the same rate.

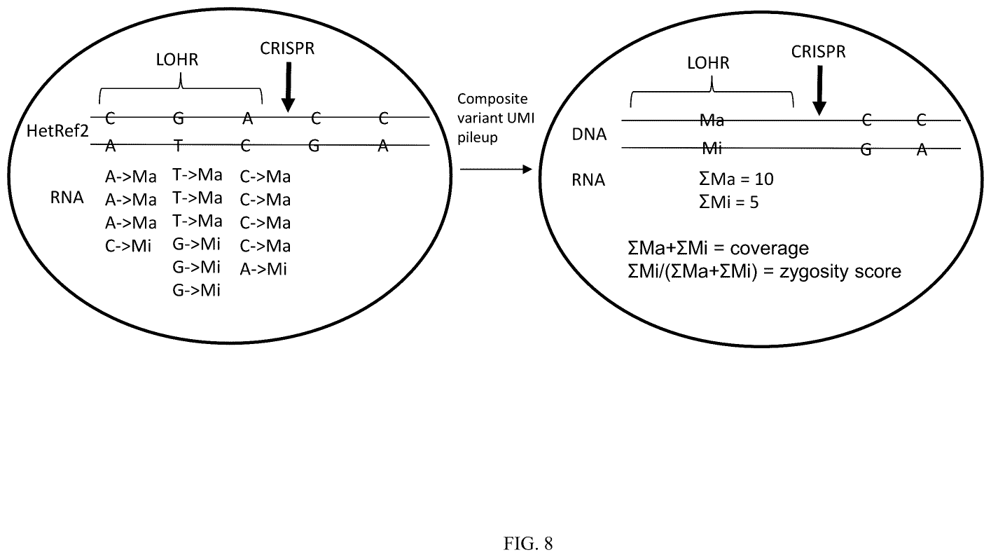

FIG. 8 illustrates how zygosity information from all eligible heterozygous variant positions of the second heterozygous variant reference in the loss of heterozygosity region were compiled to obtain the overall zygosity score and unique molecular identifier coverage for each cell; however, zygosity score=0 was not required or sufficient to determine a cell had a loss of heterozygosity because sequencing error, mapping error or both could prevent a complete loss of heterozygosity determination or overall low UMI coverage in LOHR, according to an example embodiment. Equation 1, the sum of the number of unique molecular identifiers of the more frequently detected nucleotide in each variant position, or ΣMₐ, and the number of unique molecular identifiers of the less frequently detected nucleotide in each variant position, or ΣMᵢ, were used to calculate the coverage for each cell. The heterozygous variant positions of the second heterozygous variant reference with coverage by at least 4 unique molecular identifiers were included.

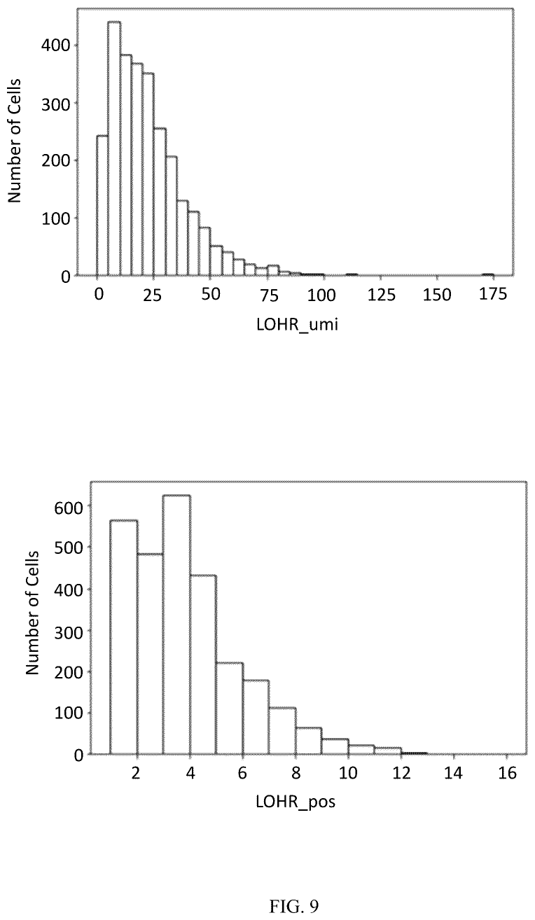

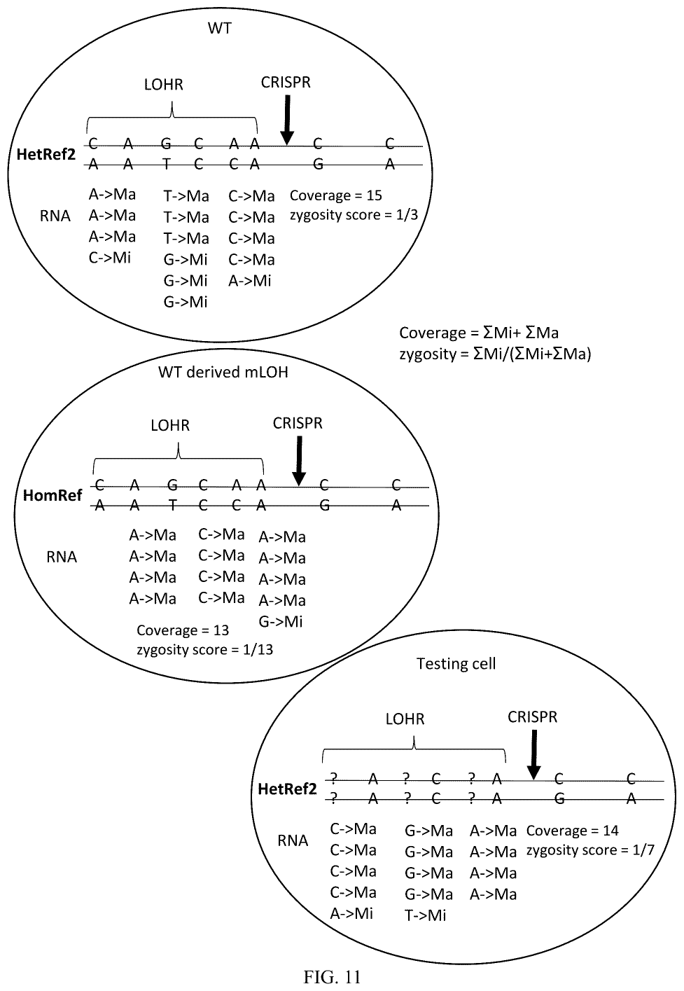

FIG. 9 shows the heterozygous variant positions of the second heterozygous variant reference that were covered by 4 or more unique molecular identifiers in the loss of heterozygosity region of wild-type cells, according to an example embodiment. The top panel of FIG. 11 shows the sum of unique molecular identifiers with coverage of at least 4 unique molecular identifiers per position in the loss of heterozygosity region in chromosome 16 of wild-type cells, according to an example embodiment. The bottom panel of FIG. 9 shows the number of heterozygous variant positions of the second heterozygous variant reference covered by at least 4 unique molecular identifiers in the loss of heterozygosity region in chromosome 16 of wild-type cells, according to an example embodiment. The coverage of each cell, which can be determined using Equation 1, was used to determine whether a cell was zygosity assessable.

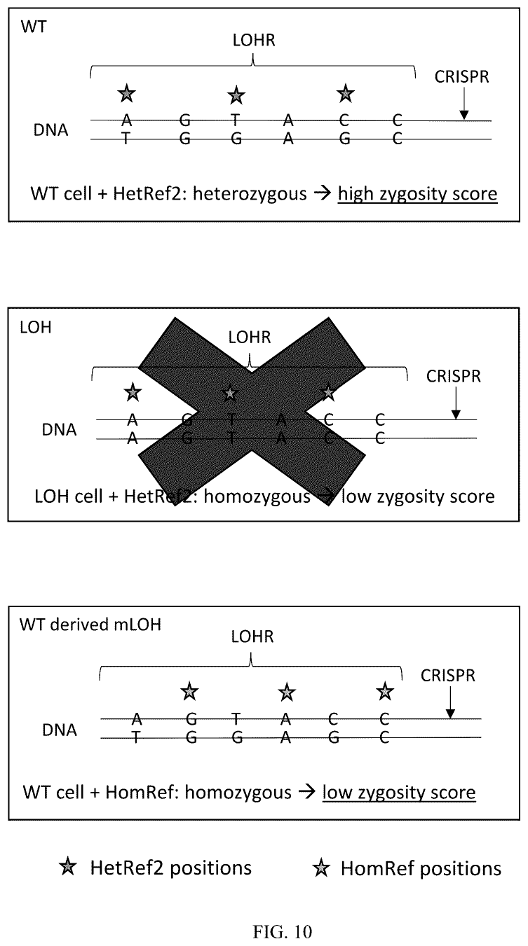

FIG. 10 illustrates how the homozygous reference including the homozygous chromosome coordinates was generated by adding about 100 to or subtracting about 100 from each heterozygous variant chromosome coordinate in the second heterozygous variant reference of wild-type cells, according to an example embodiment. Mock loss of heterozygosity cells were digitally generated from wild-type cells by calculating the coverage and zygosity score of the homozygous positions in the homozygous reference using Equation 1 and Equation 2, respectively. The zygosity scores of wild-type and mock loss of heterozygosity cells, which are the same as the coverage-weighted average of Mi/(Mi+Ma) across all eligible heterozygous variant positions, were used to train the simple logistic regression model for predicting loss of heterozygosity.

FIG. 11 illustrates how the impurity information from all eligible heterozygous variant positions of the second heterozygous variant reference in the loss of heterozygosity region of wild-type cells, loss of heterozygosity cells and mock loss of heterozygosity cells were compiled to form an artificial composite variant with unique molecular identifier coverage equal to the sum of the number of unique molecular identifiers of the more frequently detected nucleotide in each variant position, or ΣMₐ, and the number of unique molecular identifiers of the less frequently detected nucleotide in each variant position, or ΣMᵢ, as seen in Equation 1, before determining the minimum coverage to deem a cell zygosity accessible, according to an example embodiment.

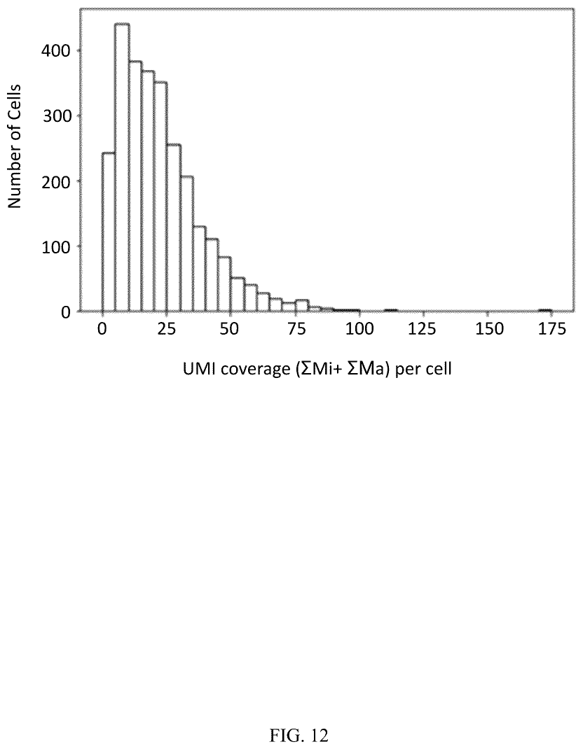

FIG. 12 shows the sum of the unique molecular identifiers in the loss of heterozygosity region on chromosome 16 of wild-type cells for which there were at least 4 unique molecular identifiers for each variant position, according to an example embodiment. The minimum unique molecular identifier coverage requirement, which is the sum of unique molecular identifier coverage across any positions registered in the second heterozygous variant reference that have at least 4 unique molecular coverage per position, should be applied to selecting wild-type and mock loss of heterozygosity cells for model training, selecting wild-type, mock loss of heterozygosity and real loss of heterozygosity cells in model independent validation and selecting cells from testing samples for loss of heterozygosity prevalence detection.

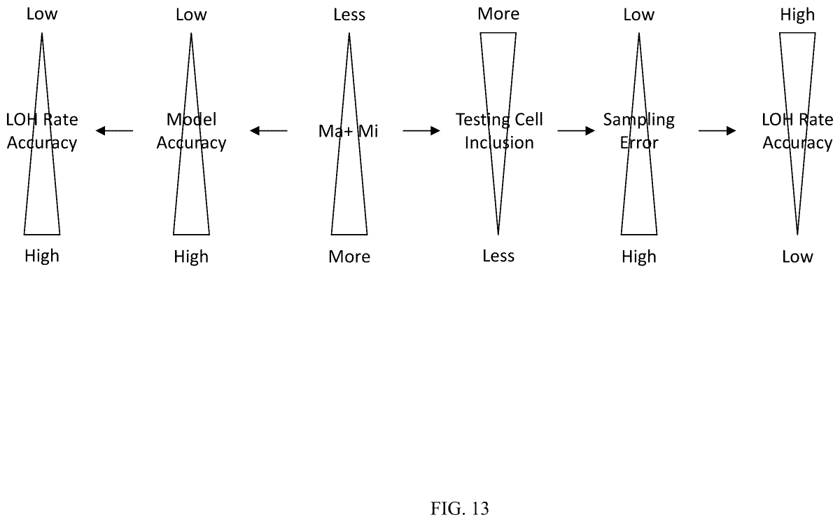

The accuracy of a model for detecting the loss of heterozygosity in individual cells and sampling error in testing samples can affect the accuracy of the model for detecting the prevalence of loss of heterozygosity. FIG. 13 illustrates that coverage requirement for the minimum number of unique molecular identifiers can affect model accuracy and sampling error in the opposite ways, according to an example embodiment. FIG. 13 also illustrates that the accuracy of a model for predicting the loss of heterozygosity for individual cells increases as the minimum unique molecular identifier coverage requirement increases, thereby potentially causing the accuracy of a model for predicting the prevalence of the loss of heterozygosity to increase, according to an example embodiment.

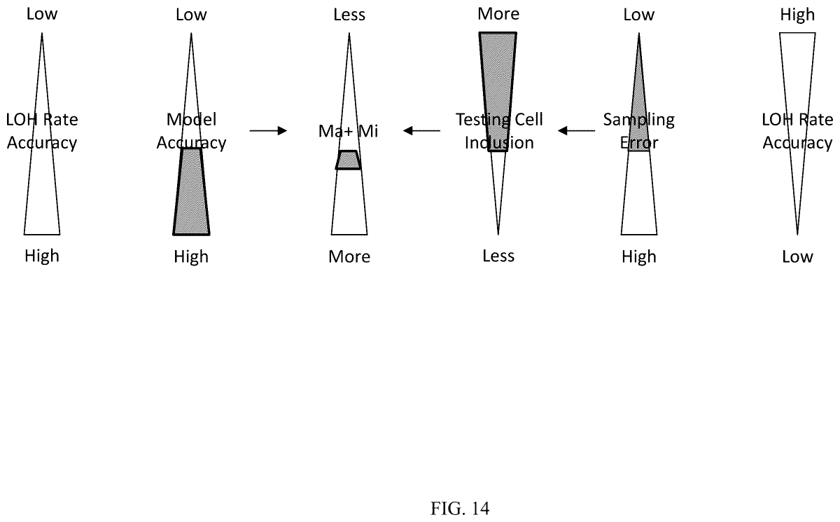

However, FIG. 13 also illustrates that the number of testing cells that pass the minimum unique molecular identifier coverage requirement decreases and the sampling error of a model increases as the minimum unique molecular identifier coverage requirement increases, thereby potentially causing the accuracy of a model for predicting the prevalence of the loss of heterozygosity to decrease, according to an example embodiment. FIG. 14 illustrates how dynamically determining the coverage requirement for determining that a cell was zygosity assessable could maximize model accuracy and minimize sampling error, according to an example embodiment.

Table 3 shows the sample size calculations for the study that were obtained using Equation 8 and the assumed prevalence (p), precision (d) and 90% of confidence (Z). The data in Table 3 indicate a precision of between about 20% and about 25% of an assumed prevalence (heterozygosity rate). It was concluded that over 1,000 zygosity assessable cells were required for an initial loss of heterozygosity prevalence estimation.

TABLE 3

| | | Assumed Loss of Heterozygosity Rate | | | | | | | | |
|---|---|---|---|---|---|---|---|---|---|---|
| | | 0.02 | 0.03 | 0.04 | 0.05 | 0.10 | 0.15 | 0.20 | 0.25 | 0.30 |
| Precision | 0.005 | 2122 | 3150 | 4156 | 5141 | 9742 | 13801 | 17319 | 20295 | 22731 |
| | 0.01 | 530 | 787 | 1039 | 1285 | 2435 | 3450 | 4330 | 5074 | 5683 |
| | 0.02 | 133 | 197 | 260 | 321 | 609 | 863 | 1082 | 1268 | 1421 |
| | 0.03 | 59 | 87 | 115 | 143 | 271 | 383 | 481 | 564 | 631 |
| | 0.04 | 33 | 49 | 65 | 80 | 152 | 216 | 271 | 317 | 355 |
| | 0.05 | 21 | 31 | 42 | 51 | 97 | 138 | 173 | 203 | 227 |
| | 0.06 | 15 | 22 | 29 | 36 | 68 | 96 | 120 | 141 | 158 |
| | 0.07 | 11 | 16 | 21 | 26 | 50 | 70 | 88 | 104 | 116 |
| | 0.08 | 8 | 12 | 16 | 20 | 38 | 54 | 68 | 79 | 89 |

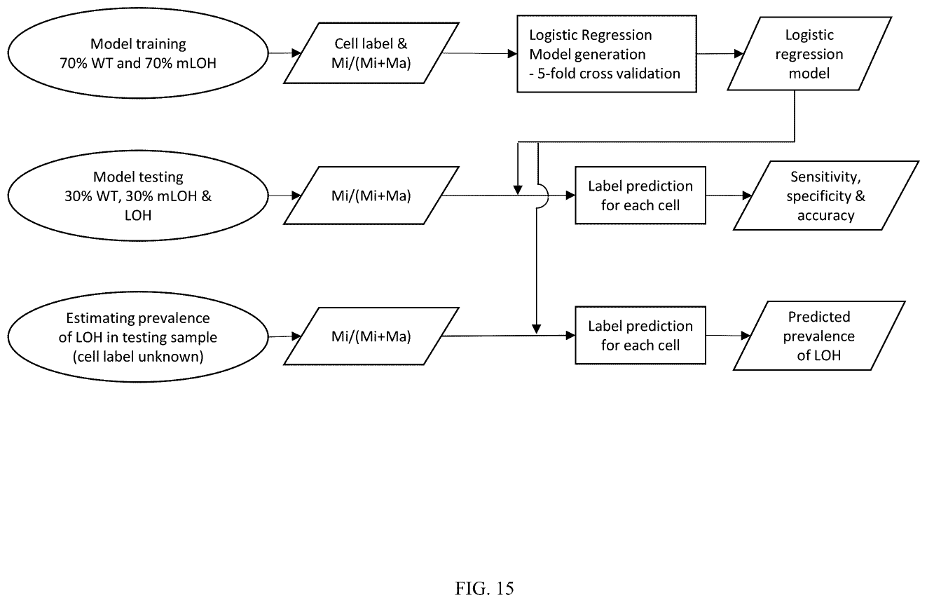

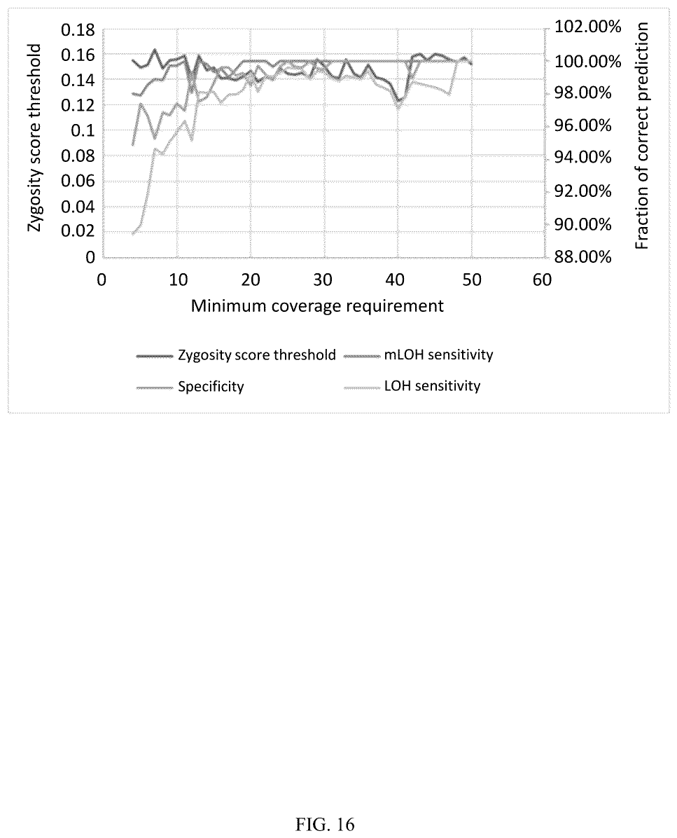

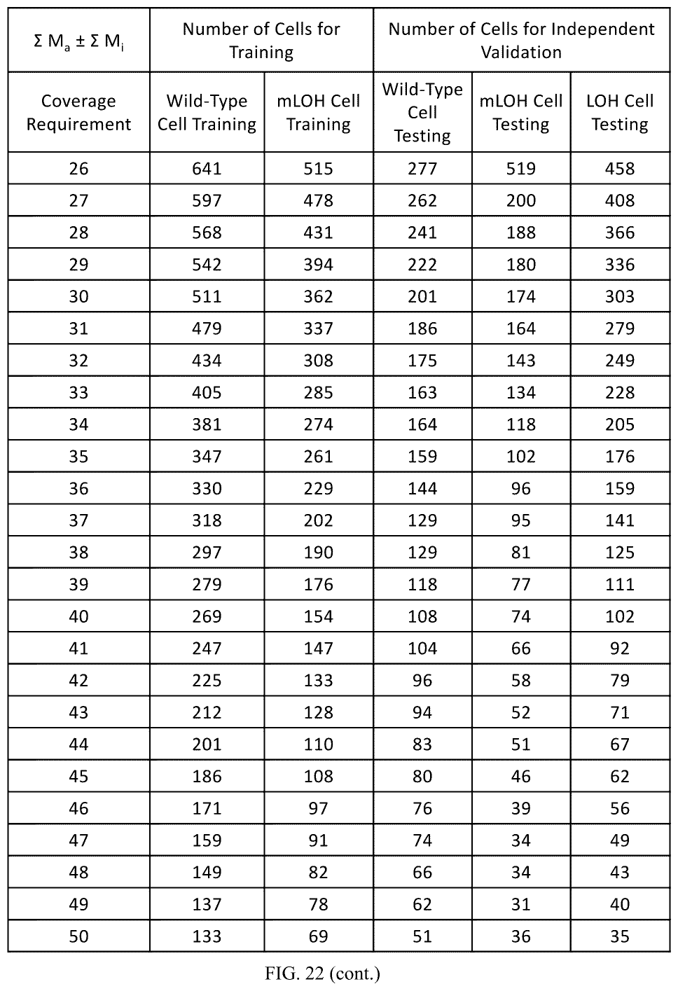

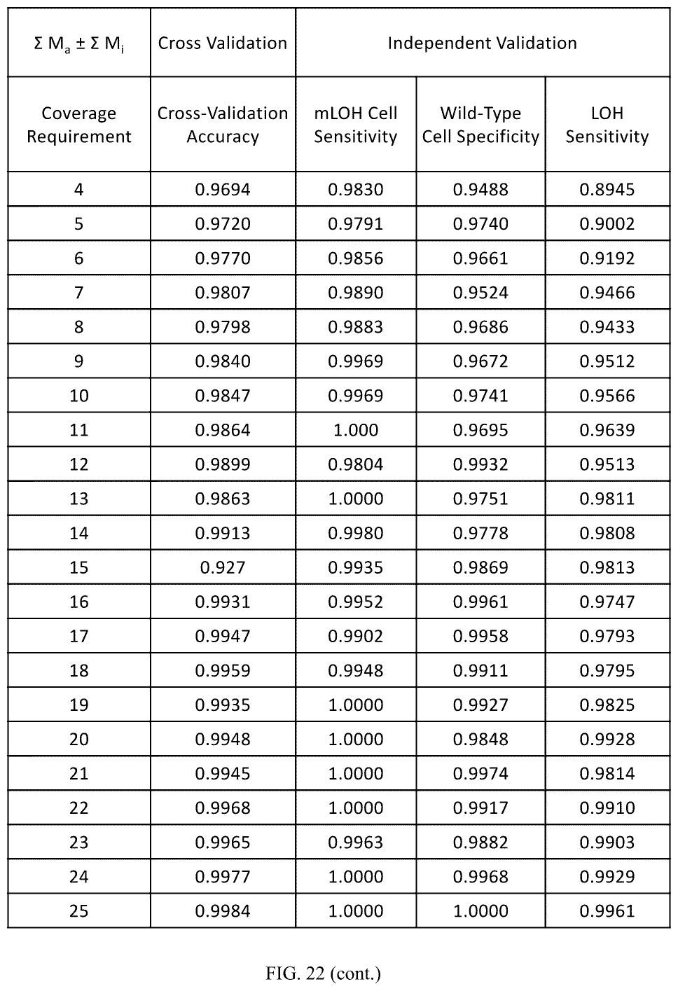

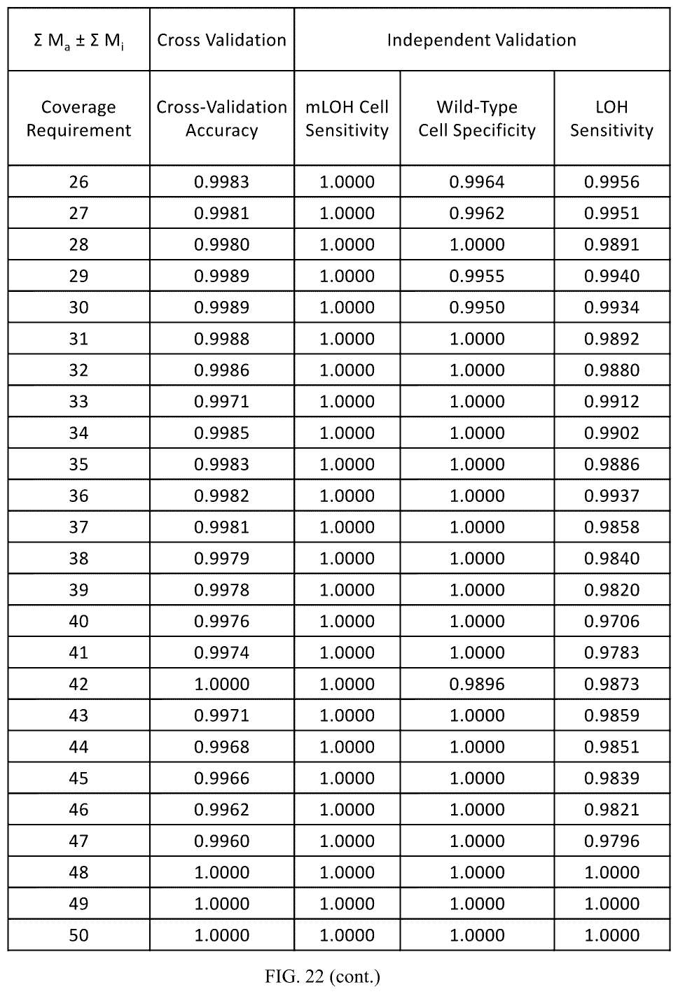

FIG. 15 illustrates the outline of an example embodiment for generating, testing and using a logistic regression model for predicting loss of heterozygosity in individual cells and the prevalence of loss of heterozygosity in a population of cells. FIG. 16 shows how the zygosity score threshold and fraction of correct predictions correlate with the unique molecular identifier coverage requirement for each row in FIG. 22, where each row is a model instance that was trained and validated using cells that passed the indicated unique molecular identifier coverage requirement, according to an example embodiment.

FIG. 16 shows that the zygosity score threshold and fraction of correct predictions generally increased as the unique molecular identifier coverage requirement increased. Model instances were trained using 70% wild-type and mock loss of heterozygosity cells and tested on the remaining 30% wild-type and mock loss of heterozygosity cells, as well as loss of heterozygosity cells. The model was generated using scikit-learn with LogisticRegressionCV classifier in Python. Stratified five-fold cross validation selected the best L2-regularization hyper-parameter. The final model was generated through fitting the entire training set with the best regularization hyper-parameter from cross-validation. A higher coverage requirement resulted in higher model accuracy, sensitivity and specificity. The model trained by wild-type and mock loss of heterozygosity cells could accurately predict zygosity for wild-type, mock loss of heterozygosity and loss of heterozygosity cells.

The specificity of the major class prediction could significantly affect the overall prediction accuracy for severely imbalanced samples where one class member is significantly more prevalent than another. For example, if the true loss of heterozygosity prevalence is 5% and the loss of heterozygosity prediction sensitivity and specificity of the model are 95% and 100%, respectively, the loss of heterozygosity prevalence would be predicted to be 4.75%, which is close to 5%; however, if the loss of heterozygosity prediction sensitivity and specificity of the model are 100% and 95%, respectively, the loss of heterozygosity prevalence would be predicted to be 9.75%, which is very different from 5%.

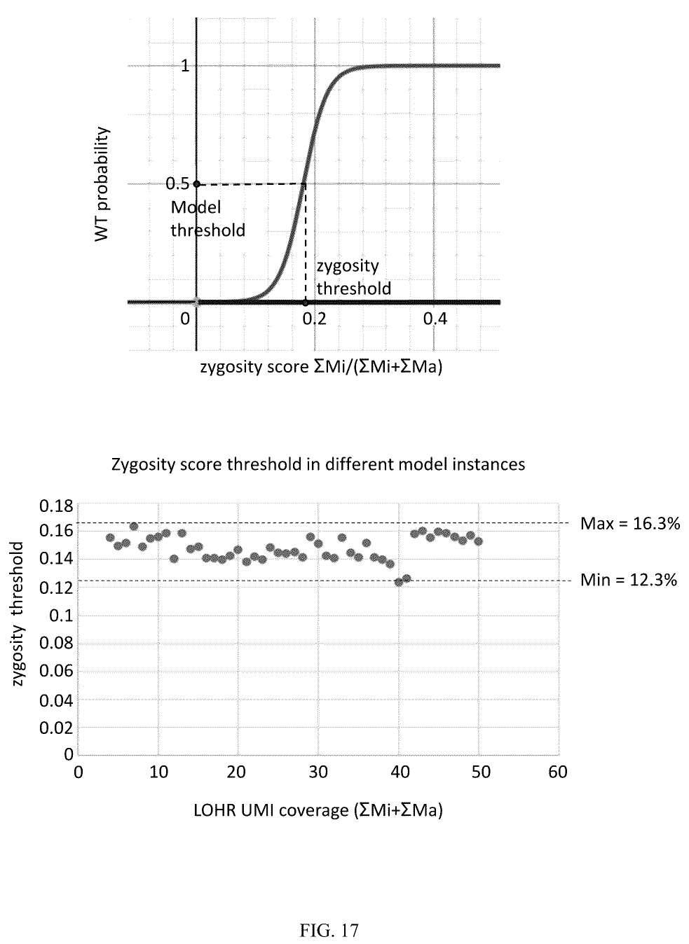

The top panel of FIG. 17 shows that the WT (wild-type) probability threshold of each model instance for predicting whether cells were wild-type cells or loss of heterozygosity cells was set to 0.5, according to an example embodiment. Cells with a WT probability that was at least 0.5 were predicted to be wild-type cells. Cells with a WT probability that was less than 0.5 were predicted to be loss of heterozygosity cells. The bottom panel of FIG. 17 shows zygosity score threshold of each logistic regression prediction model instance generated using various unique molecular identifier coverage requirements, according to an example embodiment.

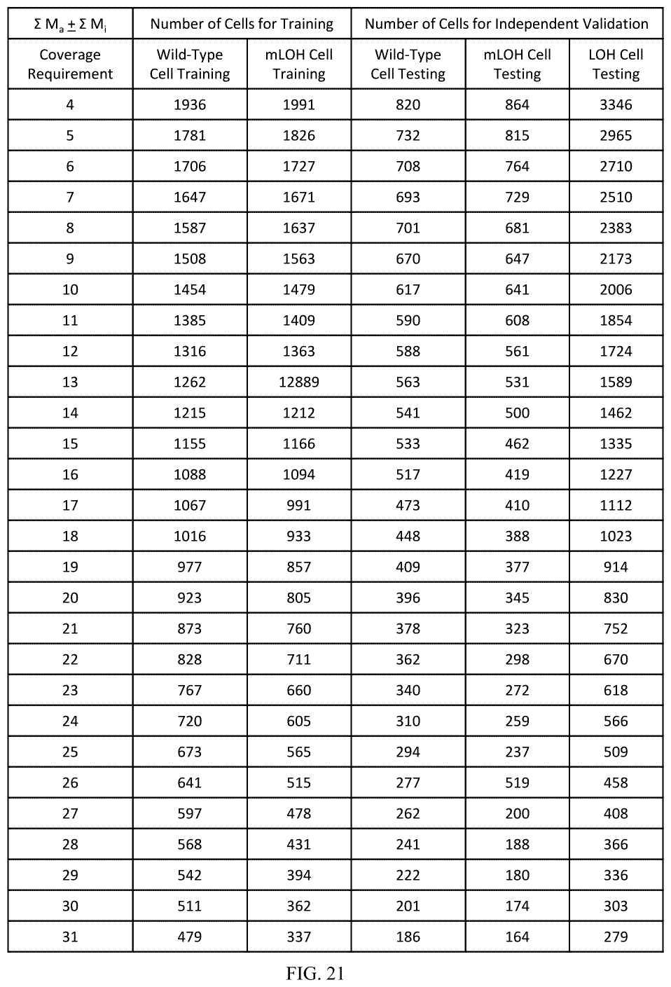

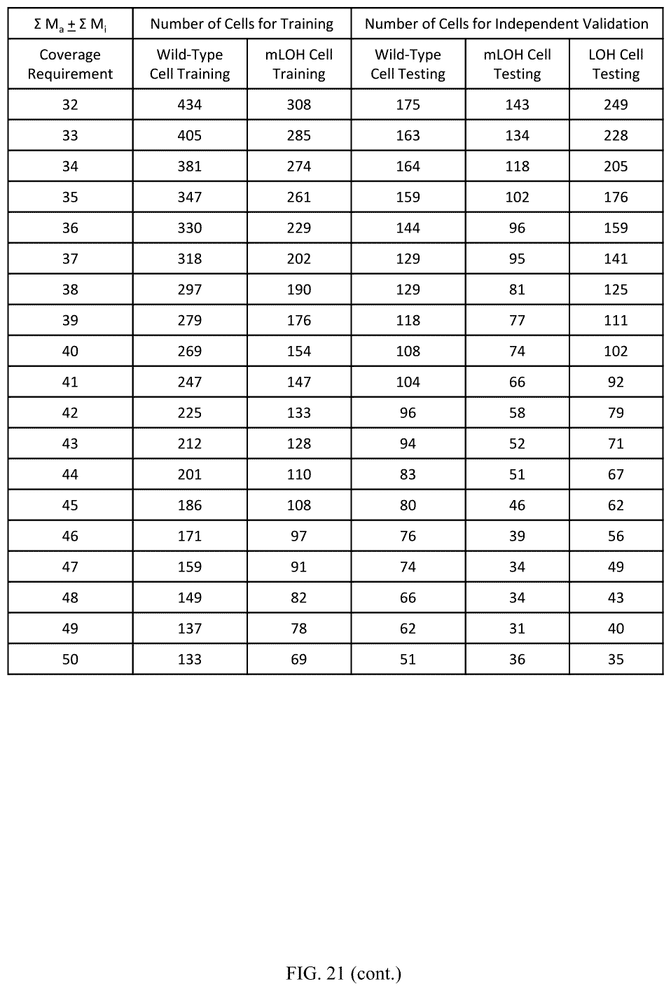

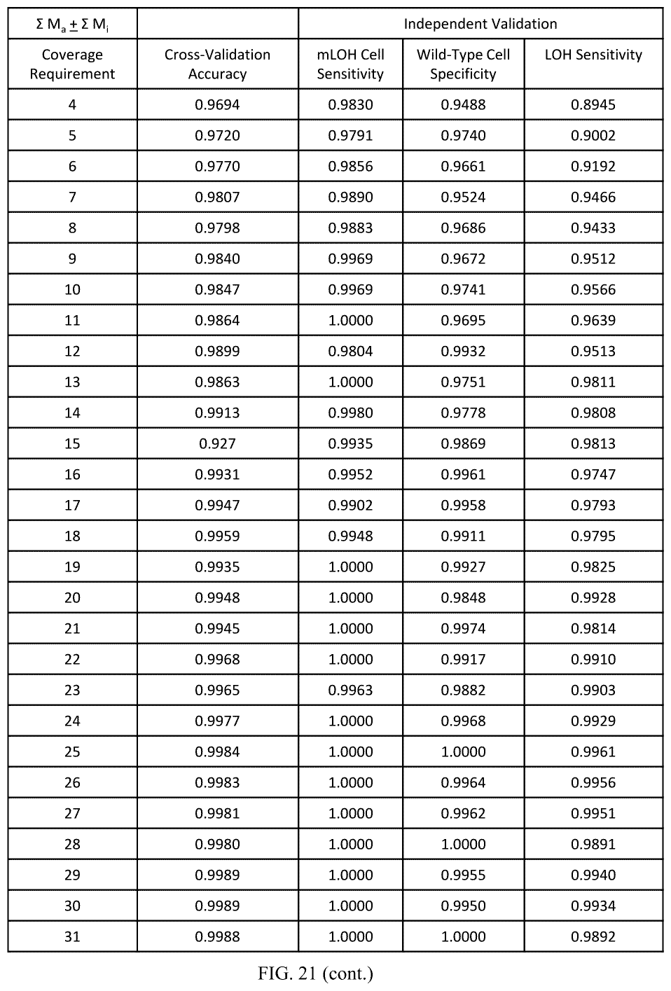

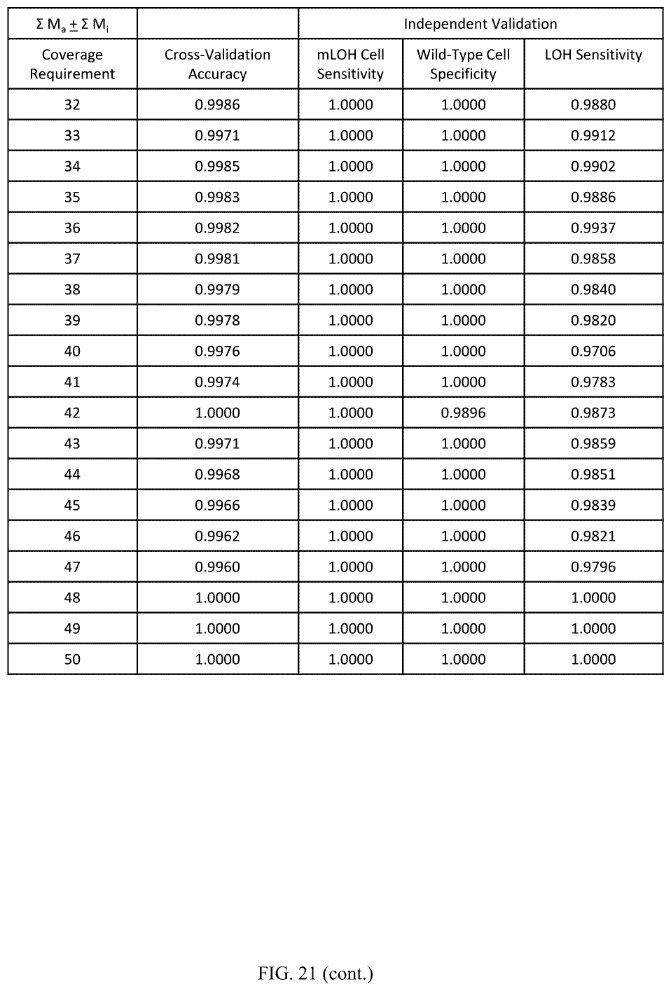

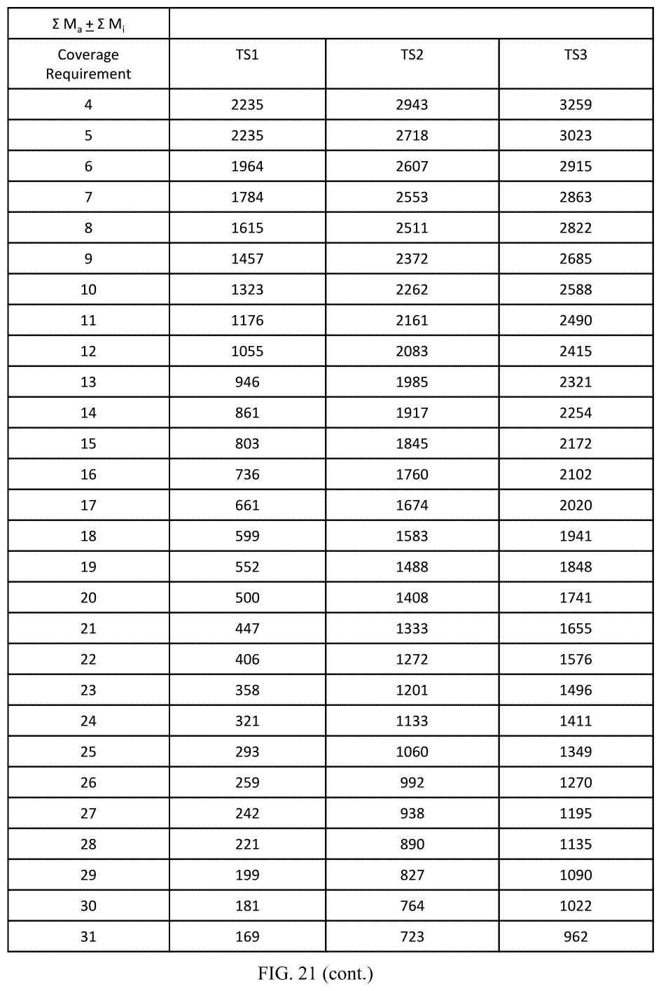

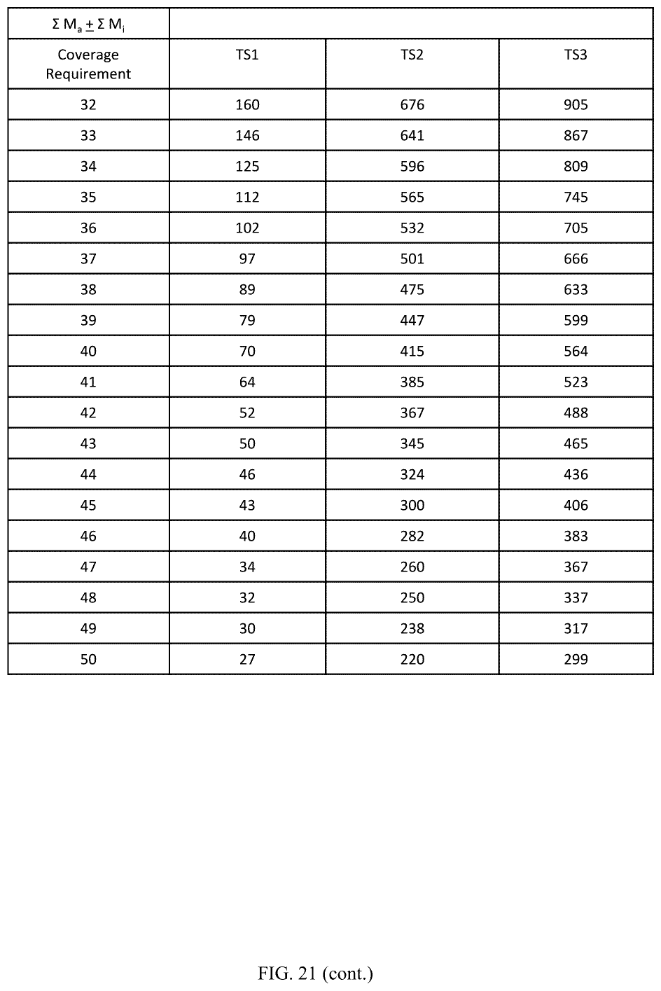

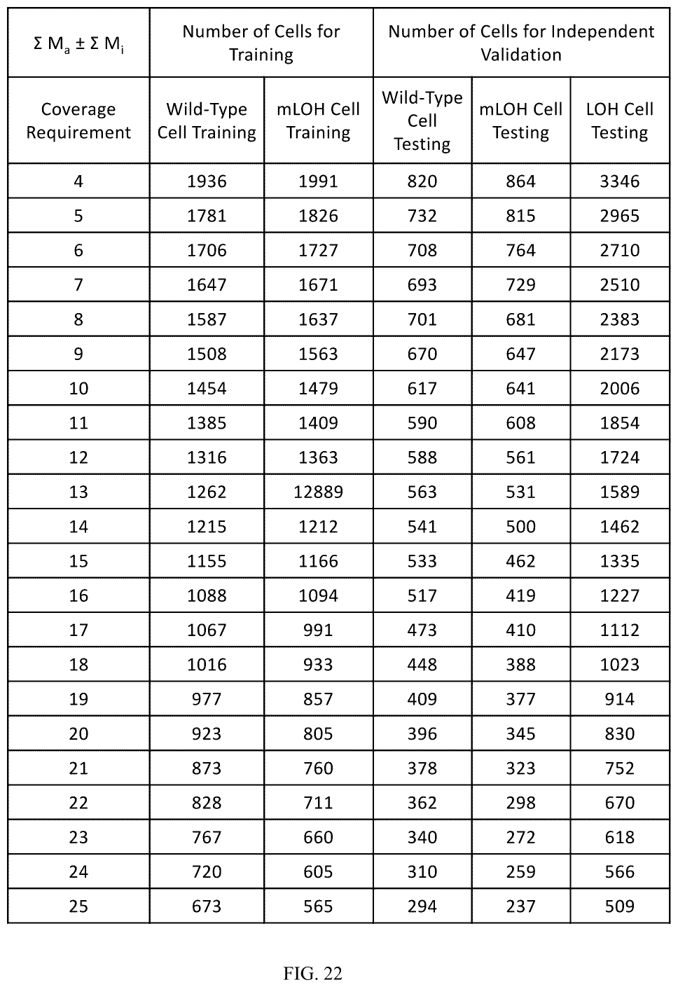

A minimum of 1,000 cells were included in each testing sample to minimize sampling error. The number of zygosity assessable cells that met the 1,000-cell minimum requirement for testing sample 1 (TS1), testing sample 2 (TS2), and testing sample 3 (TS3) are shown in FIG. 21. For test sample 1, any coverage requirement between (and including) 4 UMI to 12 UMI would satisfy the minimum 1,000 zygosity assessable cell requirement. Among the model instances using these coverage requirements, the most accurate model instance used 12 UMI as the minimum coverage requirement. For test sample 2, any coverage requirement between (and including) 4 UMI to 25 UMI would satisfy the minimum 1,000 zygosity assessable cell requirement. For test sample 3, any coverage requirement between (and including) 4 UMI to 30 UMI would satisfy the minimum 1,000 zygosity assessable cell requirement. Among the model instances using these requirements for test sample 2 or test sample 3, the most accurate model instance used a minimum coverage requirement of 25 UMI for both test sample 2 and 3.

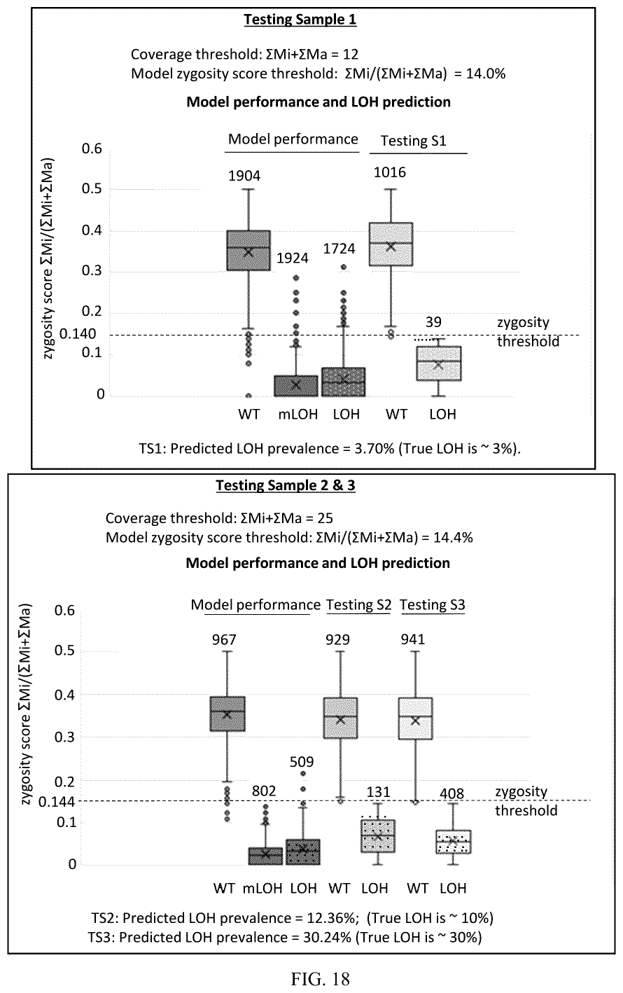

FIG. 18 shows the loss of heterozygosity prevalence predicted for the three testing samples using the model instances shown in lines 4-24 of FIG. 21 for test sample 1 and model instances shown in line 25 of FIG. 21 for testing samples 2 and 3 and listed at the top of the top and bottom panels in FIG. 18, according to an example embodiment. The top panel in FIG. 18 and FIG. 21 show that the coverage and model zygosity score thresholds used for testing sample 1 were 12 UMI and 14.0%, respectively, according to an example embodiment.

The top panel in FIG. 18 shows that the loss of heterozygosity prevalence predicted for testing sample 1 using the indicated model instance was 3.70% and that the true loss of heterozygosity prevalence for testing sample 1 was about 3%, according to an example embodiment. The bottom panel in FIG. 18 and FIG. 21 show that the coverage and model zygosity score thresholds used for testing samples 2 and 3 were 25 UMI and 14.4%, respectively, according to an example embodiment.

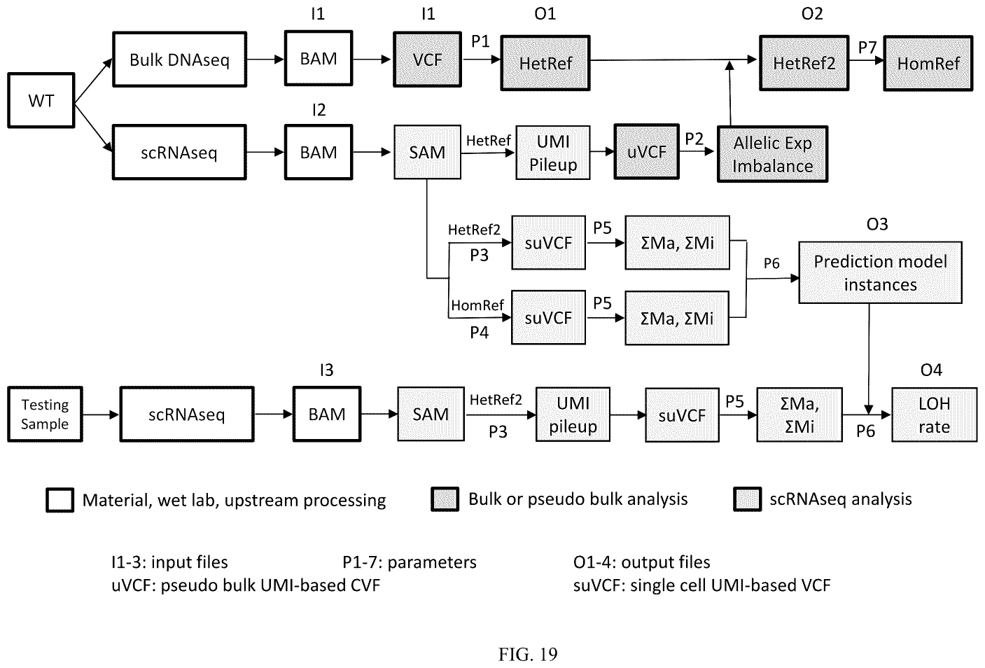

The bottom panel in FIG. 18 shows that the loss of heterozygosity prevalence predicted for testing sample 2 using the indicated model instance was 12.36% and that the true loss of heterozygosity prevalence for testing sample 2 was about 10%, according to an example embodiment. The bottom panel in FIG. 18 also shows that the loss of heterozygosity prevalence predicted for testing sample 3 using the indicated model instance was 30.24% and that the true loss of heterozygosity prevalence for testing sample 3 was about 30%, according to an example embodiment. FIG. 19 illustrates an example workflow of the disclosure, which worked because such workflow: 1) removed allelic expression imbalanced variant positions from zygosity assessment; 2) used unique molecular identifiers instead of conventional read count for zygosity assessment; 3) used a coverage of at least 4 unique molecular identifiers as a threshold for a variant position in a loss of heterozygosity region to be included in zygosity assessment; 4) used cell zygosity score as a zygosity measurement for loss of heterozygosity model building and loss of heterozygosity prediction; 6) used mock loss of heterozygosity cells that were derived from wild-type cells by generating zygosity scores and coverages from unique molecular identifiers mapped to homozygous positions in the loss of heterozygosity region; and 7) established a minimum coverage requirement for a cell to be eligible for loss of heterozygosity model training, wherein zygosity assessment was determined by generation of an accurate loss of heterozygosity prediction model that could predict loss of heterozygosity in individual cells and enough cells were included in the testing sample to avoid large sampling error. In the figure, SAM stands for Sequence Alignment/Map format file, BAM stands for binary SAM file, and VCF stands for variant calling file.

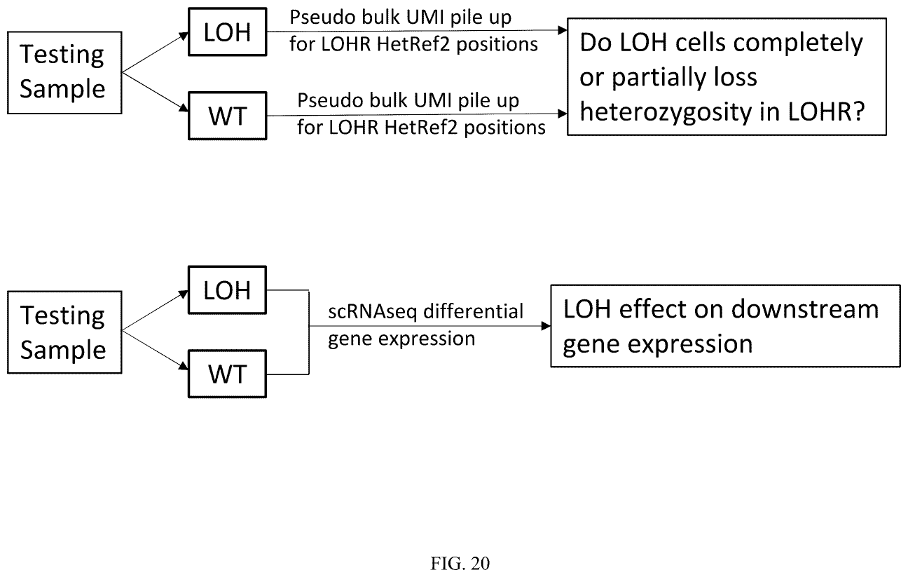

FIG. 20 illustrates how the example workflow of the disclosure can be used to determine whether loss of heterozygosity cells completely or partially loses heterozygosity in the loss of heterozygosity region and the effect of loss of heterozygosity on downstream gene expression. The top panel of FIG. 20 illustrates how the example workflow of the disclosure can analyze pseudo bulk unique molecular identifier pile up of the heterozygous variant positions of the second heterozygous variant reference in the loss of heterozygosity region for wild-type cells and loss of heterozygosity cells to determine whether loss of heterozygosity cells completely or partially loses heterozygosity in the loss of heterozygosity region. The bottom panel of FIG. 20 illustrates how the example workflow of the disclosure can analyze single-cell RNA sequencing differential gene expression data from wild-type cells and loss of heterozygosity cells to determine the effect of loss of heterozygosity on downstream gene expression.

1. A system for predicting a prevalence of loss of heterozygosity (LOH) in a target population of cells, the system comprising:
at least one memory storing computer-executable instructions; and
at least one processor in communication with the at least one memory, wherein the at least one processor is configured to execute the computer-executable instructions to:
receive genetic data for a first reference population of cells;
sequence the genetic data for the first reference population of cells to obtain first reference data, the first reference data comprising chromosome identification, nucleotide coordinates, nucleotide composition, or combinations thereof;
identify and remove heterozygous variant positions having imbalanced allelic expression in the first reference data based on single cell RNA sequencing data generated from a second reference population of cells to generate second reference data;
establish a set of homozygous positions that are a predetermined number of nucleotide distances away from each variant position of the second reference data to generate third reference data;
map identifiers for each cell of the target population of cells to the second reference data to generate first mapped identifiers;
map identifiers for each cell of the second reference population of cells to the second reference data and/or the third reference data to generate second mapped identifiers;
apply one or more inputs to a supervised machine learning model, the one or more inputs comprising the first mapped identifiers for each cell of the target population of cells and the second mapped identifiers for each cell of the second reference population of cells, the model being previously trained using historical data, the historical data comprising mapped identifiers for each cell of the target population of cells and their corresponding LOH; and
receive one or more outputs from the model, at least one of the one or more outputs including an LOH for the target population of cells,
thereby predicting the prevalence of LOH in the target population of cells;
update the historical data to include the genetic data for the target population of cells and the corresponding one or more outputs; and
re-train the model using the updated historical data.

2. The system of claim 1, wherein the target population of cells comprise genome-edited cells.

3. The system of claim 2, wherein the genome edited cells comprise CRISPR-edited cells.

4. The system of claim 1, wherein the target population of cells comprise cancer cells.

5. The system of claim 1, wherein the first reference population of cells comprise the same genotype as wild-type cells of the first reference population of cells.

6. The system of claim 1, wherein the at least one processor is configured to execute the computer-executable instructions to sequence the genetic data using bulk DNA sequencing.

7. The system of claim 1, wherein the second reference population of cells comprise the cells untreated with genome-editing tools.

8. The system of claim 1, wherein the identifiers comprise UMIs.

9. The system of claim 1, wherein the model comprises a logistic regression model.

10. A computer-implemented method for predicting a prevalence of loss of heterozygosity (LOH) in a target population of cells, the method comprising:
at least one memory storing computer-executable instructions; and
at least one processor in communication with the at least one memory, wherein the at least one processor is configured to execute the computer-executable instructions to:
receiving genetic data for a first reference population of cells;
sequencing the genetic data for the first reference population of cells to obtain first reference data, the first reference data comprising chromosome identification, nucleotide coordinates, nucleotide composition, or combinations thereof;
identifying and removing heterozygous variant positions having imbalanced allelic expression in the first reference data based on single cell RNA sequencing data generated from a second reference population of cells to generate second reference data;
establishing a set of homozygous positions that are a predetermined number of nucleotide distances away from each variant position of the second reference data to generate third reference data;
mapping identifiers for each cell of the target population of cells to the second reference data to generate first mapped identifiers;
mapping identifiers for each cell of the second reference population of cells to the second reference data and/or the third reference data to generate second mapped identifiers;
applying one or more inputs to a supervised machine learning model, the one or more inputs comprising the first mapped identifiers for each cell of the target population f cells and the second mapped identifiers for each cell of the second reference population of cells, the model being previously trained using historical data, the historical data comprising mapped identifiers for each cell of the target population of cells and their corresponding LOH; and
receiving one or more outputs from the model, at least one of the one or more outputs including an LOH for the target population of cells,
thereby predicting the prevalence of LOH in the target population of cells;
updating the historical data to include the genetic data for the target population of cells and the corresponding one or more outputs; and
re-training the model using the updated historical data.

11. The computer-implemented method of claim 10, wherein the target population of cells comprise genome-edited cells.

12. The computer-implemented method of claim 11, wherein the genome-edited cells comprise CRISPR-edited cells.

13. The computer-implemented method of claim 10, wherein the target population of cells comprise cancer cells.

14. The computer-implemented method of claim 10, wherein the first reference population of cells comprise the same genotype as wild-type cells of the first reference population of cells.

15. The computer-implemented method of claim 10, the method comprises sequencing the genetic data using bulk DNA sequencing.

16. The computer-implemented method of claim 10, wherein the second reference population of cells comprise cells untreated with genome-editing tools.

17. The computer-implemented method of claim 10, wherein the identifiers comprise UMIs.

18. The computer-implemented method of claim 10, wherein the model comprises a logistic regression model.

19. At least one non-transitory computer-readable storage media having computer-executable instructions embodied thereon, wherein when executed by at least one processor, the computer-executable instructions cause the at least one processor to:
receive genetic data for a first reference population of cells;
sequence the genetic data for the first reference population of cells to obtain first reference data, the first reference data comprising chromosome identification, nucleotide coordinates, nucleotide composition, or combinations thereof;
identify and remove heterozygous variant positions having imbalanced allelic expression in the first reference data to generate second reference data;
establish a set of homozygous positions that are a predetermined number of nucleotide distances away from each variant position of the second reference data to generate third reference data;
map identifiers for each cell of a target population of cells to the second reference data to generate first mapped identifiers;
map identifiers for each cell of the second reference population of cells to the second reference data and/or the third reference data to generate second mapped identifiers;
apply one or more inputs to a supervised machine learning model, the one or more inputs comprising the first mapped identifiers for each cell of the target population of cells and the second mapped identifiers for each cell of the second reference population of cells, the model being previously trained using historical data, the historical data comprising mapped identifiers for each cell of the target population of cells and their corresponding LOH; and
receive one or more outputs from the model, at least one of the one or more outputs including an LOH for the target population of cells,
thereby predicting the prevalence of LOH in the target population of cells;
update the historical data to include the genetic data for the target population of cells and the corresponding one or more outputs; and
re-train the model using the updated historical data.

20-40. (canceled)
